(19) United States
(12) Patent Application Publication
(10) Pub. No.: US 2025/0369950 A1
(43) Pub. Date: Dec. 4, 2025

(54) Electrokinetic Active Particles for Multimodal Biosensing

(71) Applicant: THE REGENTS OF THE UNIVERSITY OF COLORADO, A BODY CORPORATE, Denver, CO (US)

(72) Inventors: Charles W. Shields, Longmont, CO (US); Cooper Thome, Boulder, CO (US)

(21) Appl. No.: 19/128,525

(22) PCT Filed: Nov. 9, 2023
(86) PCT No.: PCT/US2023/079237

§ 371 Date: May 8, 2025

Related U.S. Application Data
(60) Provisional application No. 63/424,032, filed on Nov. 9, 2022.

(51) Int. Cl. G01N 33/487; G01N 33/543; G01N 33/553

(52) U.S. Cl. CPC G01N 33/48707; G01N 33/54313; G01N 33/553

(57) ABSTRACT

This invention is related to the field of biosensors. In particular, devices and methods are described which enable the quantification of biomolecules by virtue of particle speed driven by induced-charge electrophoresis. For example, a range of biomolecules can be simultaneously detected in a highly sensitive manner from a library (e.g., combination) of active particles having different shapes or fluorescent signatures. Such biomolecules are related to conditions including, but not limited to, cancer biomarkers, viral infection biomarkers, toxins, pesticides, and small molecule drugs.

### STATEMENT OF GOVERNMENT INTEREST

This invention was made with government support under CBET 2143419 awarded by the National Science Foundation and 1R21AI154266 awarded by the National Institutes of Health. The government has certain rights in the invention.

### FIELD OF THE INVENTION

This invention is related to the field of biosensors. In particular, devices and methods are described which enable the quantification of biomolecules by virtue of particle speed driven by induced-charge electrophoresis. For example, a range of biomolecules can be simultaneously detected in a highly sensitive manner from a library (e.g., combination) of active particles having different shapes. Such biomolecules are related to conditions including, but not limited to, cancer biomarkers, viral infection biomarkers, toxins, pesticides, and small molecule drugs.

### BACKGROUND

Detection of biomolecules—a process known as biosensing—has always played a significant role in a myriad of applications including patient diagnosis, disease management, and environmental monitoring. While there exist a vast number of biomolecule detection approaches, most incorporate three main elements: i) a target biomolecule, or biomarker, being detected (e.g., small molecules, proteins, nucleic acids); ii) a recognition element that specifically interacts with and identifies the target biomolecule; and iii) a transducer that converts target biomolecule recognition into a measurable signal.¹⁻⁶

Further, these conventional particle-based assays are generally classified by their signal output, with the most commonly demonstrated classes being electrochemical and optical. Thus, signals generated by recognition events are generally limited to changes in: i) electrical signal,^{14,15}; ii) solution color,^{12,16} and iii) fluorescence.^{17,18} Measuring these conventional signals require complex (e.g., non-standard) equipment including: i) ultraviolet-visible spectrophotometers; and ii) fluorimeters.¹²⁻¹⁸ For many, access to this specialized equipment is often limited.

What is needed in the art are detection systems that generate simplified, easily measured signals from electroactive particles. As disclosed herein, a combination of electrokinetic particles and biosensing provides a long felt need in medicine for improved workflows in detecting biomarkers.

### SUMMARY OF THE INVENTION

This invention is related to the field of biosensors. In particular, devices and methods are described which enable the quantification of biomolecules by virtue of particle speed driven by induced-charge electrophoresis. For example, a range of biomolecules can be simultaneously detected in a highly sensitive manner from a library (e.g., combination) of active particles having different shapes. Such biomolecules are related to conditions including, but not limited to, cancer biomarkers, viral infection biomarkers, toxins, pesticides, and small molecule drugs. In one embodiment, the present invention contemplates a composition comprising: i) a plurality of first electrokinetic active particles (EAPs) having a first shape and attached or bound to a first biomolecule recognition element; and ii) a plurality of second EAPs having a second shape and attached or bound to a second biomolecule recognition element. In one embodiment, the first and second biomolecule recognition element includes, but is not limited to, an antibody, an antibody fragment, a protein, an affibody, an aptamer, an oligonucleotide, an antigen, an enzyme, a cell receptor and/or a ligand. In one embodiment, the first and second biomolecule recognition element has a binding affinity to a biomarker. In one embodiment, the biomarker includes, but is not limited to, osteoprotegerin (OPG), human epidermal growth factor receptor 2 (HER2), interleukin (IL)-6, cell-free DNA (cfDNA), CD44⁺ exosomes, and circulating tumor cells (CTCs). In one embodiment, the first shape has an angular structure that is different than the second shape. In one embodiment, the angular structure of the first shape comprises an angle that is less than the second shape. In one embodiment, the angular structure of the second shape comprises an angle that is greater than that of the first shape. In one embodiment, the angular structure of the first shape comprises a 65° angle. In one embodiment, the angular structure of the second shape comprises a 115° angle. In one embodiment, the first biomolecule recognition element has a different coating density than the second biomolecule recognition element. In one embodiment, each of the plurality of first and second EAPs further comprise a metallic surface region. In one embodiment, the metallic surface region is a patch. In one embodiment, the metallic surface region includes, but is not limited to, magnetic or non-magnetic metals or conductive polymers. In one embodiment, the metallic surface region is a gold surface region. In one embodiment, the metallic surface region is dielectric. In one embodiment, the metallic surface region is attached to the first or second biomolecule recognition element. In one embodiment, the first and second EAPs are microparticles. In one embodiment, the first and second EAPs are nanoparticle. In one embodiment, the first and second EAPs are Janus particles. In one embodiment, the metallic surface is strongly polarized or polarizable. In one embodiment, the dielectric surface is weakly polarized or polarizable. In one embodiment, the metallic surface comprises a chromium layer. In one embodiment, the metallic surface comprises an electrically conducting layer of indium tin oxide. In one embodiment, the metallic surface comprises a layer comprising chromium and gold. In one embodiment, the coplanar electrode pair comprises a conductive layer and a non-conductive gap. In one embodiment, the first and second EAPs are polymeric particles. In one embodiment, the composition is label-free. In one embodiment, the first and second EAPs further comprise an antifouling layer. In one embodiment, the antifouling layer is polyethylene glycol or a glutaraldehyde/bovine serum albumin complex.

In one embodiment, the present invention contemplates a method, comprising; a) providing: i) a solution comprising a plurality of first electrokinetic active particles (EAPs) having a first shape and attached to a first biomolecule recognition element; ii) a plurality of second EAPs having a second shape and attached to a second biomolecule recognition element; iii) a electrokinetic propulsion chamber comprising a coplanar or non-coplanar electrode pair separated by an electrically insulated region; and iv) a camera mounted to a magnifying lens (e.g., microscope); b) placing an aliquot of the solution on the electrokinetic propulsion chamber; c) applying an alternating current electric field to the electrokinetic propulsion chamber; d) recording a series of images or video of the solution with the camera; and e) determining am electrokinetic motion of the first and second EAPs from the image. In one embodiment, the first biomolecule recognition element is specifically bound to a first biomarker and the second biomolecule recognition element is specifically bound to a second biomarker, wherein the first biomarker is different from the second biomarker. In one embodiment, the first and second biomolecule recognition elements include, but are not limited to, an antibody, an antibody fragment, a protein, an affibody, an aptamer, an oligonucleotide, an antigen, an enzyme, a cell receptor and/or a ligand. In one embodiment, the first and second biomarker includes, but is not limited to, an antibody or fragment thereof, an antigen, a toxin, a protein, a nucleic acid, an exosome, a small organic molecule or a whole cell. In one embodiment, the motion is an electrokinetic speed of the first EAP being slower than the second EAP. In one embodiment, the electrokinetic speed of the first EAP being faster than the second EAP. In one embodiment, the motion is an electrokinetic acceleration of the first EAP being slower than the electrokinetic acceleration of the second EAP. In one embodiment, the motion is an electrokinetic acceleration of the first EAP being faster than the electrokinetic acceleration of the second EAP. In one embodiment, the method further comprises purifying the first EAP from the second EAP. In one embodiment, the purifying comprises a magnetic purification. In one embodiment, the method further comprises identifying the first and second biomarkers. In one embodiment, the method further comprises quantifying the first and second biomarkers. In one embodiment, the first and second EAPs are microparticles. In one embodiment, the first and second EAPs are nanoparticles. In one embodiment, the first and second EAPs are Janus particles. In one embodiment, the first and second biomarkers include, but is not limited to, OPG, HER2, IL-6, cfDNA, CD44⁺ exosomes, and CTCs. In one embodiment, the first shape has an angular structure that is different than the second shape. In one embodiment, the angular structure of the first shape comprises an angle that is less than that of the second shape. In one embodiment, the angular structure of the second shape comprises an angle that is greater than that of the first shape. In one embodiment, the angular structure of the first shape comprises a 65° angle. In one embodiment, the angular structure of the second shape comprises a 115° angle. In one embodiment, the first biomolecule recognition element has a different coating density than the second biomolecule recognition element. In one embodiment, each of the plurality of first and second EAPs further comprise a metallic surface region. In one embodiment, the metallic surface region is a patch. In one embodiment, the metallic surface region includes, but is not limited to magnetic metals, non-magnetic metals or conductive polymers. In one embodiment, the metallic surface region is a gold surface region. In one embodiment, the metallic surface region is dielectric. In one embodiment, the metallic surface region is attached to the biomolecule recognition element. In one embodiment, the metallic surface is strongly polarized or polarizable. In one embodiment, the dielectric surface is weakly polarized or polarizable. In one embodiment, the metallic surface comprises a chromium layer. In one embodiment, the metallic surface comprises an electrically conducting layer of indium tin oxide. In one embodiment, the metallic surface comprises a layer comprising chromium and gold. In one embodiment, the coplanar electrode pair comprises a conductive layer and a non-conductive gap. In one embodiment, the first and second EAPs are polymeric particles. In one embodiment, the polymeric particles are label-free. In one embodiment, the electrically insulated region of the coplanar electrode pair includes, but is not limited to, glass, quartz, plastic, polydimethylsiloxane or other electrically insulating material. In one embodiment, the solution is label-free. In one embodiment, the particle is a polymeric particle. In one embodiment, the first and second EAPs further comprise an antifouling layer. In one embodiment, the antifouling layer is polyethylene glycol or a glutaraldehyde/bovine serum albumin complex.

In one embodiment, the present invention contemplates a composition comprising: i) a plurality of first electrokinetic active particles (EAPs) and attached to a biomolecule recognition element; and ii) a plurality of second EAPs and not attached to a biomolecule recognition element. In one embodiment, the biomolecule recognition element includes, but is not limited to, an antibody, an antibody fragment, a protein, an affibody, an aptamer, an oligonucleotide, an antigen, an enzyme, a cell receptor and/or a ligand. In one embodiment, the biomolecule recognition element has a binding affinity to a biomarker. In one embodiment, the biomarker includes, but is not limited to, OPG, HER2, IL-6, cfDNA, CD44⁺ exosomes, and CTCs. In one embodiment, each of the plurality of first and second EAPs further comprise a metallic surface region. In one embodiment, the metallic surface region is a patch. In one embodiment, the metallic surface region includes, but is not limited to magnetic metals, non-magnetic metals and/or conductive polymers. In one embodiment, the metallic surface region is a gold surface region. In one embodiment, the metallic surface region is dielectric. In one embodiment, the first and second EAPs are microparticles. In one embodiment, the first and second EAPs are nanoparticle. In one embodiment, the first and second EAPs are Janus particles. In one embodiment, the metallic surface is strongly polarized or polarizable. In one embodiment, the dielectric surface is weakly polarized or polarizable. In one embodiment, the metallic surface comprises a chromium layer. In one embodiment, the metallic surface comprises an electrically conducting layer of indium tin oxide. In one embodiment, the metallic surface comprises a layer comprising chromium and gold. In one embodiment, the coplanar electrode pair comprises a conductive layer and a non-conductive gap. In one embodiment, the first and second EAPs are polymeric particles. In one embodiment, the composition is label-free. In one embodiment, the first and second EAPs further comprise an antifouling layer. In one embodiment, the antifouling layer is polyethylene glycol or a glutaraldehyde/bovine serum albumin complex.

In one embodiment, the present invention contemplates a method, comprising; a) providing: i) a solution comprising at least one particle having a metallic surface and a dielectric surface; ii) a biomolecule recognition element conjugated to the metallic surface; iii) a electrokinetic propulsion chamber comprising a coplanar electrode pair separated by an electrically insulated region; and iv) a camera mounted to a magnifying lens (e.g., microscope); b) placing an aliquot of the solution on the electrokinetic propulsion chamber; c) applying an alternating current electric field to the electrokinetic propulsion chamber; d) recording a series of images or video of the solution with the camera; and e) determining the electrokinetic motion of the at least one particle from the image. In one embodiment, the at least one particle comprises a first particle conjugated to a first biomolecule recognition element. In one embodiment, the at least one particle comprises a second particle conjugated to a second biomolecule recognition element. In one embodiment, the first biomolecule recognition element is specifically bound to a first biomolecule. In one embodiment, the second biomolecule recognition element is specifically bound to a second biomolecule. In one embodiment, the first biomolecule recognition element includes, but is not limited to, a first antibody, a first antibody fragment, a first protein, a first affibody, a first aptamer, a first oligonucleotide, a first antigen, a first enzyme, a first cell receptor and/or a first ligand. In one embodiment, the second biomolecule recognition element includes, but is not limited to, a second antibody, a second antibody fragment, a second protein, a second affibody, a second aptamer, a second oligonucleotide, a second antigen, a second enzyme, a second cell receptor and/or a second ligand. In one embodiment, the first biomolecule includes, but is not limited to, an antibody or fragment thereof, an antigen, a toxin, a protein, a nucleic acid, an exosome, a small organic molecule etc. In one embodiment, the second biomolecule includes, but is not limited to, an antibody, an antigen, a toxin, a protein, a nucleic acid, an exosome, a small organic molecule etc. In one embodiment, the motion is an electrokinetic speed of the first particle being slower than the second particle. In one embodiment, the motion is an electrokinetic speed of the first particle being faster than the second particle In one embodiment, the method further comprises purifying the first particle. In one embodiment, the method further comprises purifying the second particle. In one embodiment, the method further comprises identifying the first biomolecule. In one embodiment, the method further comprises identifying the second biomolecule. In one embodiment, the method further comprises quantifying the first biomolecule. In one embodiment, the method further comprises quantifying the second biomolecule. In one embodiment, the particle is a microparticle. In one embodiment, the particle is a nanoparticle. In one embodiment, the particle is a Janus particle. In one embodiment, the metallic surface is strongly polarized or polarizable. In one embodiment, the dielectric surface is weakly polarized or polarizable. In one embodiment, the metallic surface comprises a chromium layer. In one embodiment, the metallic surface comprises an electrically conducting layer of gold or indium tin oxide. In one embodiment, the metallic surface comprises a layer comprising chromium and gold. In one embodiment, the coplanar electrode pair comprises a conductive layer and a non-conductive gap. In one embodiment, the coplanar electrode pair comprises a chromium layer. In one embodiment, the coplanar electrode pair comprises an electrically conducting layer of gold, indium tin oxide or another conductive material. In one embodiment, the electrically insulated region of the coplanar electrode pair includes, but is not limited to, glass, quartz, plastic, polydimethylsiloxane or other electrically insulating material. In one embodiment, the polymeric particle is label-free. In one embodiment, the particle is a polymeric particle. In one embodiment, the motion is an electrokinetic acceleration of the first particle being slower than the electrokinetic acceleration of the second particle. In one embodiment, the motion is an electrokinetic acceleration of the first particle being faster than the electrokinetic acceleration of the second particle.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “polymeric particle” as used herein, refers to a solid object substantially constructed of a polymer, a co-polymer and/or block polymer. For example, the polymer, co-polymer and/or block polymer may comprise between 80-99%, preferably 90-99% and most preferably 95-99% of the polymeric particle.

The term “dielectric surface” as used herein, refers to a surface comprising an electrical insulator material that is capable of becoming weakly polarized. In particular, charges do not flow through the material as they do in an electrical conductor, because dielectric materials have no loosely bound, or free, electrons that may drift through the material, but instead they shift, only slightly, from their average equilibrium positions, causing a dielectric polarization. As used herein, representative dielectric surfaces include, but are not limited to, porcelain, glass, and most plastics.

The term “polarized” as used herein, refers to a unified alignment and orientation of at least partially metallic particles in an applied electric field. In particular, a particle becomes polarized when an electric field distorts a negative cloud of electrons around a positive atomic nucleus in a direction opposite the field. This slight separation of charge makes one side of the atom somewhat positive and the opposite side somewhat negative in a uniform manner throughout the particle.

The term “biomolecule” as used herein, refers to a biological molecule present within or outside of a living system. For example, a biomolecule may belong to an organic chemical class including, but not limited to, proteins, nucleic acids, polysaccharides, or small organic molecules. Proteins may be peptides and/or polypeptides and act as enzymes, antibodies and/or a biological hormone. For example, a biological hormone includes, but is not limited to, insulin, glucagon, thyrocalcitonin, pituitary hormones and/or hypothalamic hormones.

The term “recognition element” as used herein, refers to a molecule comprising a molecular pattern and/or conformation that specifically binds to a biomolecule. For example, the biomolecule recognition element includes, but is not limited to, an antibody and fragments thereof, cell receptors, ligands, proteins, enzymes, or nucleic acids. The biomolecule recognition element may include, but is not limited to, an amino acid sequence, a nucleic acid sequence, a macromolecule and/or a small organic molecule.

The term “coplanar” as used herein, refers to two or more objects (e.g., electrodes) aligned and/or oriented within the same plane.

The term “aliquot” as used herein, refers to a predetermined volume routinely sampled from a larger (e.g., stock) volume.

The term “electrokinetic” as used herein, refers to any phenomenon that is caused by the flow of electricity.

The term “propulsion speed” or “propulsion rate” as used herein, refers to the relative motion of a particle caused by an external force. For example, the propulsion speed or rate may be caused by an electrokinetic force, where particle motion is a result of the flow of electricity (e.g., an application of an alternating current electric field). Propulsion speed or rate may be rotational or translational. For example, during rotational propulsion the particle remains stationary while during translational propulsion the particle undergoes a linear movement.

The term “propulsion acceleration” as used herein, refers to a rate of change in propulsion speed of an electrokinetic active particle. It is to be understood that even if two particles have different propulsion accelerations, they may have the same or different propulsion speeds at any given time.

The term “video” as used herein, refers to a visible impression obtained by a camera, telescope, microscope, or other device, or displayed on a computer or video screen. For example, the image may be a timelapse “digital” image from which quantitative information regarding objects within the image may be calculated. For example, electrokinetic propulsion speed of Janus particles may be calculated from digital images taken within an electrokinetic propulsion chamber.

The term “electrical permittivity” as used herein, refers to the measure of the electric polarizability of a substance (e.g., biomarker molecules, dielectric materials).

The term “electroosmosis” as used herein, refers to osmosis behavior when under the influence of an electric field.

The term “Janus particle” as used herein, refers to a particle (e.g., nanoparticles or microparticles) whose surface has two or more distinct physical properties. For example, a Janus particle may have a polystyrene surface on a first hemisphere and a chromium and/or gold surface on a second hemisphere.

The term “unitary gold-layered particle” as used herein, refers to a particle wherein the entire particle surface comprises gold.

The term “attached” or “bound” as used herein, refers to any interaction between a first molecule and a second molecule. Attachment may be reversible or irreversible. Such attachment includes, but is not limited to, covalent bonding, ionic bonding or Van der Waals forces, and the like.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term, “purified” or “isolated”, as used herein, may refer to particles or biomarkers bound to particles that have been subjected to treatment (i.e., for example, fractionation) to remove various other components, and which composition substantially retains its expressed biological activity. Where the term “substantially purified” is used, this designation will refer to a composition in which the particles or biomarkers bound to particles forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that particles or biomarkers bound to particles are the dominant species (i.e., for example, based upon DLS analysis). A purified composition is not intended to mean that all trace impurities have been removed.

As used herein, the term “substantially purified” refers to biomarkers that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “nucleic acid sequence” or “nucleotide sequence” as used herein, refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable.

The term “antibody” refers to immunoglobulin evoked in animals by an immunogen (antigen) or immunoglobulin produced by synthetic means, including but not limited to recombinant antibodies. It is desired that the antibody demonstrates specificity to epitopes contained in the immunogen. The term “polyclonal antibody” refers to immunoglobulin produced from more than a single clone of plasma cells or by synthetic means; in contrast “monoclonal antibody” refers to immunoglobulin produced from a single clone of plasma cells or by synthetic means.

The terms “specific binding” or “specifically binding” when used in reference to the interaction of a biomolecule recognition element and a biomolecule means that the interaction is dependent upon the presence of a particular structure (i.e., for example, an antigenic determinant or epitope) on a protein or other biomolecule. For example, an antibody may recognize and bind to a specific protein structure rather than to proteins in general. For example, if an antibody is specific for epitope “A”, it may attach to protein containing epitope A (or free, unlabeled A).

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “label” or “detectable label” are used herein, to refer to any composition detectable by spectroscopic, photochemical, biochemical, immunochemical, electrical, optical, or chemical means. Such labels include biotin for staining with labeled streptavidin conjugate, magnetic beads (e.g., Dynabeads®), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. If a compound or molecule is not attached or bound to a “label”, the compound or molecule is therefore designated as “label-free”.

The terms “binding component”, “molecule of interest”, “agent of interest”, “ligand” or “receptor” as used herein may be any of a large number of different molecules, biological cells or aggregates, and the terms are used interchangeably. Each binding component may be immobilized on a solid substrate and binds to an analyte being detected. Proteins, polypeptides, peptides, nucleic acids (nucleotides, oligonucleotides, and polynucleotides), antibodies, ligands, saccharides, polysaccharides, microorganisms such as bacteria, fungi and viruses, receptors, antibiotics, test compounds (particularly those produced by combinatorial chemistry), plant and animal cells, organdies or fractions of each and other biological entities may each be a binding component. Each, in turn, also may be considered as analytes if the same bind to a binding component on a surface.

The term “macromolecule” as used herein, refers to any molecule of interest having a high molecular weight. For example, some biopolymers having a high molecular weight would be comprised of greater than 100 amino acids, nucleotides, or sugar molecules long.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme, and the analyte is a substrate for the enzyme. Reactions resulting from contact between the binding agent and the analyte are also within the definition of binding for the purposes of the present invention.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence of nucleic acids. Furthermore, the molecular arrangement may comprise a lipid bilayer or other biological material.

### Abbreviations

BPT, biotin-PEG-thiol; DEP, dielectrophoresis; ICEM, induced-charge electrophoretic microsensor; ICEP, induced-charge electrophoresis; MQW, milli-Q water; PEG, polyethylene glycol; PBS, phosphate-buffered saline; PTFE, polytetrafluoroethylene; SA, streptavidin.

### BRIEF DESCRIPTION OF THE FIGURES

FIG. 1 illustrates the relationships between biomolecule concentration and electromotive speed (e.g., propulsive force).

FIG. 1A: A schematic representation of biomolecule detection by Janus particle ICEMs comprising a polystyrene (PS) surface on a first hemisphere and a gold surface on a second hemisphere. Further, the PS hemisphere is functionalized with polyethylene glycol (PEG) to reduce nonspecific adsorption of off-target matrix components and reduce particle-surface interactions. The gold hemisphere is functionalized with biotin recognition elements for capture of a target biomolecule, streptavidin (SA). As indicated, capture of SA quantifiably reduces the electrophoretic speed of particles as can be determined in visual detection methods (e.g., microscopy).

FIG. 1B: A schematic representation showing that Janus particle ICEM speed decreases in this specific case in proportion with increased biomolecule concentration.

FIG. 2 depicts a representative electrokinetic Janus particle fabrication method.

FIG. 2A: Polystyrene particles are first deposited on a glass slide as a submonolayer using a convective assembly method (left panel). The particles are then coated with chromium (Cr) and gold (Au) using electron beam evaporation (middle panel). Finally, Janus particles are mechanically scraped off the glass slide and transferred to milli-Q water (right panel).

FIG. 2B: Representative image of a submonolayer of polystyrene particles on a glass slide.

FIG. 2C: Representative scanning electron microscopy image of coated Janus particles. Yellow coloring was manually applied to highlight the approximate location of gold patches on the particles, as indicated by visually rough surfaces.

FIG. 2D: Representative fluorescence image of Janus particles in MQW. Light and dark regions of the particles are exposed polystyrene and gold patches, respectively. Due to the density of the gold patch, particles typically settle with the gold hemisphere facing downward (i.e., toward the microscope objectives). Insert: A magnified view of coated Janus particles visualizing the polymer hemisphere (light) and the gold-layered hemisphere (dark).

FIG. 2E: Scanning electron microscopy image of Janus particles with a gold-layered hemisphere. Gold hemispheres are seen as a rough surface with light gray on the dark PS surface. The image was taken using a Hitachi SU3500 SEM in a secondary electron imaging mode at an acceleration voltage of 15 kV.

FIG. 3 presents exemplary data showing photomicrographs of electrokinetic active particle propulsion and tracking.

FIG. 3A: A representative illustration of an electrokinetic particle propulsion chamber. A particle aliquot (in an aqueous solution) is placed between two gold electrodes and within the indicated hydrophobic boundary and two PTFE spacers.

After applying a coverslip, the aqueous particle aliquot spreads to the hydrophobic boundary edge. An electric field is generated by applying an alternating current (AC) square wave from a function generator having electrical connectivity to the chamber by the leads and copper tape.

FIG. 3B: A series of superimposed images from a time-lapse recording of a single particle undergoing ICEP within a propulsion chamber over the course of 6 seconds. As is characteristic of ICEP, the particles propel perpendicular to the applied electric field and in the direction of the polystyrene hemisphere.

FIG. 3C: A first frame of captured videos with the electric field “Off” with superimposed particle paths over the course of 4.0 seconds.

FIG. 3D: A first frame of captured videos with the electric field “On” with superimposed particle paths over the course of 4.0 seconds.

FIG. 3E: A schematic of representative tracking criteria. For example, in this study, only particles that exhibited linear movement 0°+15° of normal to the applied field in either direction were tracked.

FIG. 4 presents one strategy for ICEM biomolecular functionalization.

FIG. 4A: A schematic representation of an ICEM functionalization protocol. Blank Janus particles are incubated in a solution of PEG-amine and 1-ethyl-3-(3-dimethylaminopropyl) carbodiimide (EDC) in 2-(N-morpholino) ethanesulfonic acid (MES) buffer for 2 hours, during which PEG-amine is covalently bound to carboxyl groups on the PS surface through carbodiimide chemistry. The particles are washed 3-4 times and resuspended in a solution of biotin-PEG-thiol and mixed overnight, enabling coupling of biotin-PEG-thiol through thiol-gold bonding.

FIG. 4B: Exemplary data showing mean ICEM electrokinetic speeds with, and without, bound PEG on a Janus particle PS hemisphere. Janus particles with a PEG-functionalized PS hemisphere displayed greater speeds, relative to those without, likely due to decreased interactions with the chamber walls. N=27 for both conditions. Error bars indicate standard error of the mean.

FIG. 4C: Exemplary data of a BPT concentration curve showing mean ICEM electrokinetic speeds. Each bar represents Janus particle PS hemispheres that were identically functionalized with PEG but conjugated with different concentrations of BPT. All particles, including the control, were incubated in a PBS solution and subsequently washed into MQW 4 times, with only concentration of BPT in the PBS solution differing between groups. No condition led to significantly different speeds compared to that of the control (N>25). Error bars indicate standard error of the mean.

FIG. 5 presents exemplary data of a ICEM biomolecule detection study.

FIG. 5A: A schematic representation of general biomolecule detection protocol. ICEMs are incubated for 30 minutes in solutions spiked with streptavidin (SA) and are subsequently washed 4 times. Particles are then placed in an electrokinetic propulsion chamber and positioned on a camera-equipped microscope. After an electric field is engaged, video recordings are made of particle motion which are analyzed to quantify mean particle speeds.

FIG. 5B: Exemplary data of normalized mean ICEM speeds after incubation with SA-spiked solutions with concentrations ranging from 0-2×10⁴ nM SA. A decrease in particle speed was observed at a concentration of 1 nM SA, though this decrease was not significant (P=0.08). All data points above 1 nM SA differed significantly from the control (0 nM SA) (P<0.05). Particles reached a minimum speed at SA=100 nM. Experimental data was fit to a 4 parameter logistic curve (4PL) curve model to generate a curve fit (N>21 for all conditions). Error bars indicate standard error of the mean.

FIG. 5C: Exemplary data showing representative particle path tracks of three ICEMs each from both the control (no SA) and 2×10⁴ nM SA conditions. Tracks indicate both x- and y-particle position over the course of 5.48 seconds.

FIG. 5D: Exemplary data showing normalized mean speed of particles incubated in 1000 nM SA (N=5), as well as no SA (N=11), 1000 nM blocked SA (e.g., an SA-biotin complex) (N=5), and free biotin (N=7) as controls. The speed of particles incubated with 1000 nM SA was significantly lower than that of the control conditions (p<10-5 relative to the 0 nM SA control). These results indicate that decreases in ICEM speed in the biomolecule detection experiments are due exclusively to specific capture of SA, rather than nonspecific adsorption. Error bars indicate standard error of the mean.

FIG. 6 presents exemplary data showing the programmability of an ICEM-based assay to capture alternative biomolecules through antibody recognition and enhance sensitivity through reduced particle concentration.

FIG. 6A: Schematic representation of the capture of OVA by anti-OVA ICEMs.

FIG. 6B: Normalized mean speed of ICEMs incubated in 1.1 μM OVA (N=12) compared to a 0 μM OVA control (N=10). The speed of anti-OVA ICEMs incubated with OVA was significantly lower than that of the 0 μM OVA control (p<0.05).

FIG. 6C: Schematic representation of assay tunability by varying ICEM number. As the number of ICEMs per sample decreases, sensitivity increases by increasing the concentration of SA on the surfaces of particles.

FIG. 6D: Normalized mean speed of ICEMs incubated in 0.1 nM SA at standard (2.5×10⁵) and reduced (1×10⁴) ICEM numbers (N=14 and N=6, respectively) as well as no SA (N=7) and 0.1 nM BSA (N=15) as controls. The speed of ICEMs incubated in 0.1 nM SA at the reduced ICEM number was significantly lower than that of the 0 nM SA control (p<0.05). Error bars in (B) and (D) indicate standard error of the mean.

FIG. 7 presents exemplary concepts of electrokinetic particle biosensors.

FIG. 7A: Particle propulsion in alternating current (AC) electric fields arising from their asymmetric surface conductivity.

FIG. 7B: Specific accumulation of biomarkers onto particle surface gold patches slows propulsion.

FIG. 7C: Multiplexed detection enabled by barcoded particles of unique shape with unique bioaffinity patches.

FIG. 8 presents exemplary electrokinetic particle spinner devices.

FIG. 8A: Schematic illustrations of an exemplary electrokinetic particle spinner.

FIG. 8B: Image of microfabricated EAPs that spin upon their central axis.

FIG. 8C: Indium tin oxide (ITO)-coated plates form a chamber for stimulating particles.

FIG. 8D: Velocity of the particles in B across varied applied field frequencies in the ICEP regime at 6.4×10⁵ V²/cm².

FIG. 9 presents exemplary electrokinetic biosensors.

FIG. 9A: A Janus sphere with captured anti-OVA biomarkers.

FIG. 9B: Particle tracking data.

FIG. 9C: Average particle speed (p<0.05).

FIG. 10 illustrates one embodiment of an EAP fabrication method.

FIG. 10A: An SU-8 photoresist template.

FIG. 10B: Alignment lithography to create small openings over specific regions of the particles.

FIG. 10C: Metal deposition performed to deposit: i) a 5 nm adhesive layer of chromium (Cr); ii) a 20 nm layer of cobalt (Co) (to enable magnetic separation); and a 100 nm conductive layer of gold (Au).

FIG. 10D: Solvents separate the two photoresist layers and particles removed by shear forces.

FIG. 10E: Antifouling coating conjugation to polymer areas and biorecognition motifs appended to the Au layer.

FIG. 10F: Particles purified using a magnetic column and stored until use.

FIG. 11 presents one illustration of a model for EAP surface geometry.

FIG. 12 presents representative examples of antifouling coatings.

FIG. 12A: Polymerized oligoethylene glycol and sulfobetaine methacrylate to reduce fouling by minimizing protein unfolding.

FIG. 12B: crosslinked BSA to reduce biofouling by steric repulsion.

FIG. 13 presents exemplary shape-encoded EAP biosensors. A panel of unique particle shapes is shown wherein each EAP has a different angular structure. Each unique EAP shape is modified with a different biorecognition element to enable capture of a different biomarker, e.g., from left to right: HER2 (65° angular structure), OPG (90° angular structure), and cfDNA (115° angular structure).

### DETAILED DESCRIPTION OF THE INVENTION

This invention is related to the field of biosensors. In particular, devices and methods are described which enable the quantification of biomolecules by virtue of particle speed driven by induced-charge electrophoresis. For example, a range of biomolecules can be simultaneously detected in a highly sensitive manner from a library (e.g., combination) of active particles having different shapes. Such biomolecules are related to conditions including, but not limited to, cancer biomarkers, viral infection biomarkers, toxins, pesticides, and small molecule drugs.

Detection of biomolecules has long been a platform for patient diagnosis, environmental monitoring, and a myriad of other applications. Recently, nano- and microparticle-based detection has been explored for improving traditional detection assays. Small scale assays improved the technology by reducing required sample volumes and assay times, as well as enhancing assay sensitivity and tunability.

Among these particle-based approaches, active particle-based assays were proposed that couple particle motion to biomolecule concentration that expanded assay accessibility through a simplified signal output. However, these methods suffered a major disadvantage by requiring secondary labeling, which complicates these assays and introduces additional points of error.

The data presented herein demonstrate an improved method for microparticle motion-based, label-free biomolecule detection. In one embodiment, the present invention contemplates an induced-charge electrophoretic microsensor (ICEM) to be used in a method to capture, detect and/or measure concentrations of biomolecules (e.g., streptavidin (SA)). In one embodiment, the biomolecule detection comprises a direct signal transduction mediated by ICEM speed suppression to measure ICEMs at nanomolar concentrations. Although it is not necessary to understand the mechanism of an invention, it is believed that the disclosed improved active particle-based biomolecule detection method is rapid, simple, and label-free.

In one embodiment, the present invention contemplates a simple and versatile particle-based platform that simultaneously detects a plurality of different biomarkers. In one embodiment, the simultaneous detection of the plurality of different biomarkers takes less time than conventional detection devices and methods. In one embodiment, the simultaneous detection of the plurality of different biomarkers has similar or improved sensitivity than conventional detection devices and methods.

In one embodiment, the present invention contemplates a composition comprising an ICEM-based particle comprising a plurality of conductive bioaffinity domains. In one embodiment, a conductive bioaffinity domain comprises a biomolecule recognition element conjugated to a metal surface. In one embodiment, the biomolecule recognition element has a specific affinity for a biomolecule. Although it is not necessary to understand the mechanism of an invention, it is believed that conductive bioaffinity domains propel particles within an alternating current (AC) electric field at a proportional rate to a biomarker concentration. In one embodiment, the proportional rate is a negative proportional rate. In other words, as the biomarker concentration increases the induced propulsion rate of the ICEM decreases. In one embodiment, the proportional rate is a positive proportional rate. In other words, as the biomarker concentration increases the induced propulsion rate of the ICEM increases. In one embodiment, the present invention contemplates a method providing a particle-based platform for separating a mixture of different biomarkers based upon the respective concentration of each specific biomarker.

Preliminary data has shown that non-equilibrium propulsion of particles can be regulated by size, placement and/or mismatch in their respective electrical permittivity between the conductive domains and the dielectric bodies of particles. For example, when energized by AC electric fields, the asymmetric polarizability of each particle generates an asymmetric fluid flow by induced-charge electroosmosis. These asymmetric fluid flow results in a directional propulsive force.

The data presented herein demonstrates an improvement in induced-charge electroosmosis utilization by biorecognition motif conjugation to particle conductive domains that create conductive bioaffinity domains configured to capture specific biomarkers. For example, biorecognition motifs are described herein which capture specific biomarkers from whole blood or other biofluids without non-specific binding of other biomolecules. As biomarkers attach to their respective conductive bioaffinity domains (e.g., constructed as metallic patches on a particle hemisphere), their relative electrical permittivity decreases, thus slowing particle locomotion.

In one embodiment, the present invention contemplates a method comprising purifying particles (e.g., ICEMs) from blood or other biofluids. In one embodiment, the method further comprises transferring the purified particles to an electroactive chamber. In one embodiment, the method further comprises stimulating the purified particles by an AC electric field generated by the electroactive chamber. In one embodiment, the method further comprises recording particle electroactive motion by microscopic visualization. In one embodiment, the recording further comprises simultaneous multimodal detection of the particle electroactive motion. In one embodiment, the particles are of different fluorescence intensity. In one embodiment, the particles are of different shapes. Although it is not necessary to understand the mechanism of an invention, it is believed that particle velocities are determined and then correlated with biomarker concentrations through independently established standard calibration curves.

Although it is not necessary to understand the mechanism of an invention, it is believed that ICEMs have exemplary advantages over conventional biosensing assays that encompass: i) a faster workflow as compared to current gold standard detection devices and assays (e.g., ELISA); and ii) the simultaneous detection of multiple biomarkers.

### I. Conventional Active Particle Detection Systems

In recent years, those skilled in the art have developed numerous approaches to detect biomolecules using both active and inactive nano- and microparticle-based systems. Compared to non-particle-based biomolecule detection assays, such as traditional enzyme-linked immunosorbent assays, particle-based assays provided enhanced detection of biomolecules by improving sample mixing, reducing required sample volumes, enabling efficient purification of biomolecules from samples, and imparting additional assay tunability.⁷⁻¹³

Indeed, a number of groups have demonstrated active-particle-based systems in which biomolecule concentration is correlated with particle motion. By coupling concentration and motion in this manner, detection assay results can be determined by more available measurement techniques such as optical microscopy, mobile phone imaging or direct visualization (e.g., microscopy).

Despite this, in a majority of the previously disclosed motion-based detection approaches, biomolecule concentration and motion are only indirectly linked. For example, target particle capture or detection does not innately lead to signal production. Instead, intermediary additional components such as enzymatic, antibody-particle, or metallic labels are required to produce a change in motion.¹⁹⁻²⁴ Introduction of such additional component represent distinct disadvantages by creating inherently complicated systems and introduces new failure modes. Often, these additional components necessitate extra assay steps thereby increasing assay time. In fact, few groups have even attempted to develop a label-free, particle motion-based sensing systems much less one that includes a specific recognition element.²⁵

### II. Electrokinetic Bioactive Molecules Detection Systems

In one embodiment, the present invention discloses a method comprising simple, label-free, active particle-based biomolecule detection. In one embodiment, the method comprises a specific capture of target biomolecules by differently shaped particles, wherein each group of particles defined by a specific shape are analyzed for changes in electrophoretic particle speed (e.g., the output signal). In one embodiment, the method comprises a specific capture of target biomolecules by particles at different concentrations, wherein each of the different concentrations of particles directly influence electrophoretic particle speed (e.g., the output signal).

In one embodiment, the present invention contemplates an induced-charge electrophoretic microsensor (ICEM) comprising a Janus particle. In one embodiment, the Janus particle comprises gold and polystyrene. In one embodiment, the Janus particle comprises magnetic material. In one embodiment, the Janus particle comprises a biomolecule recognition element. In one embodiment, the biomolecule recognition element binds to a target biomolecule.

In one embodiment, the biomolecule recognition element is biotin. In one embodiment, the target biomolecule is streptavidin (SA).

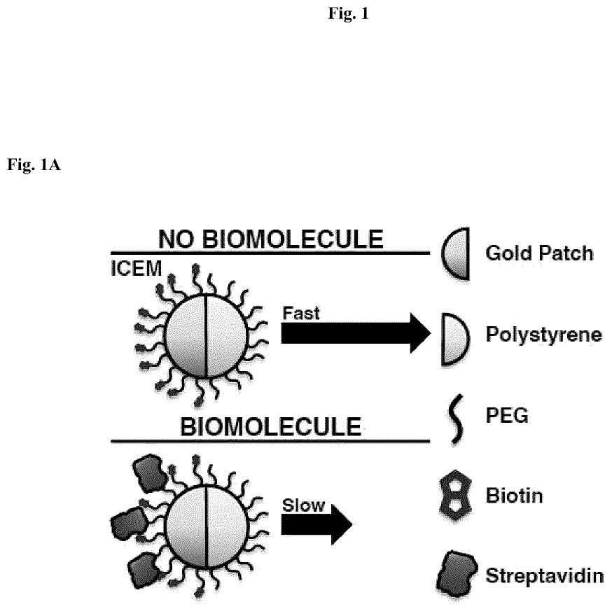

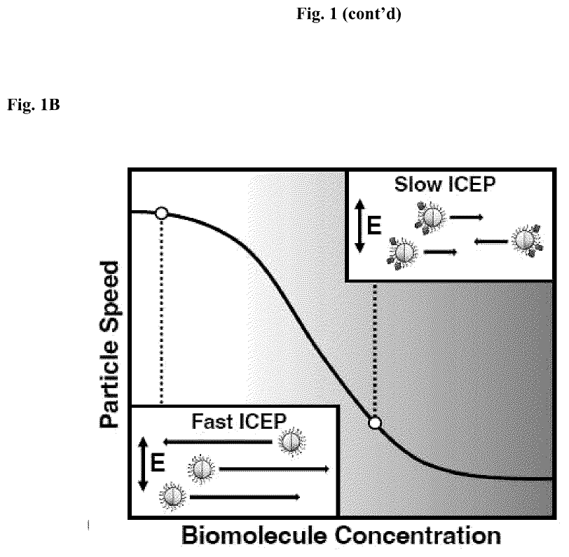

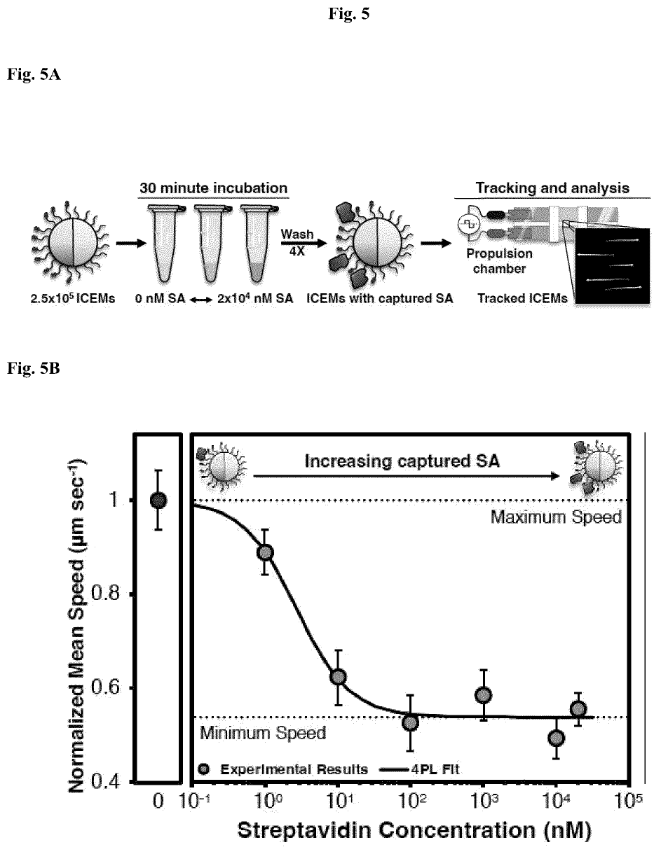

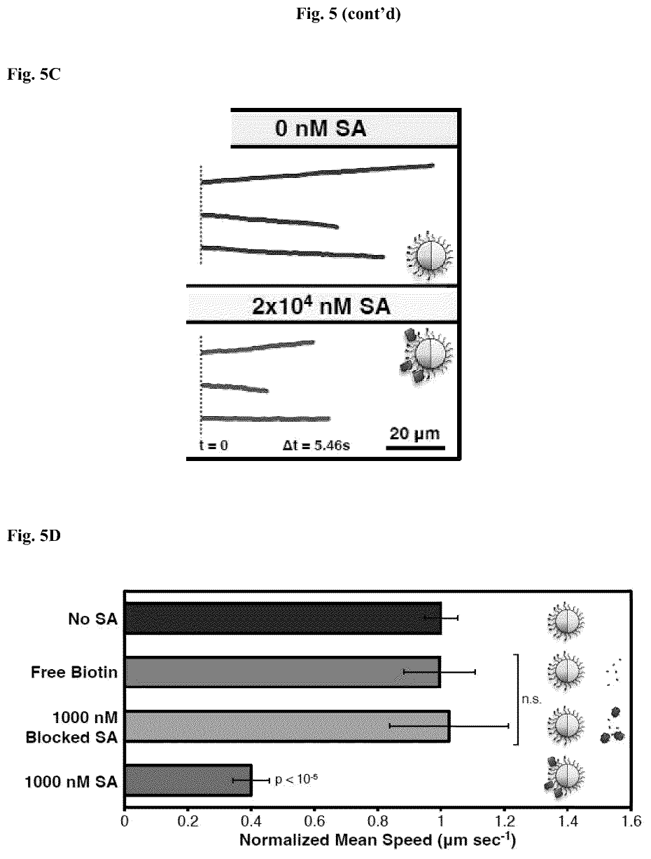

For example, a specific capture of SA by a biotin-ICEM generates direct signal transduction in the form of ICEM electrokinetic speed suppression. See, FIG. 1A. A video recorded by optical microscopy was analyzed showing that ICEM electrokinetic speed decreases in an SA concentration-dependent manner, See, FIG. 1B and FIG. 5B. These data show a similar phenomenon as observed in traditional quantitative biomolecule detection assays.⁴

### A. Induced-Charge Electrophoresis

Induced-charge electrophoresis (ICEP) refers to a process by which particles propel in an alternating current (AC) electric field due to asymmetric electroosmotic flows originating from a corresponding asymmetry in particle polarizability and/or geometry.²⁶ In the case of a spherical, metallodielectric Janus particle, ICEP stems from a disparity in the polarizability of each hemisphere of the particle: a first hemisphere that is metallic and highly polarizable and a second hemisphere that is dielectric and weakly polarizable.^{27,28} When a uniform AC field (e.g., preferably in the kHz frequency range) is applied to a spherical Janus particle submerged within a liquid the particle reorients by dielectrophoresis (DEP). This reorientation aligns the hemisphere interface parallel with the electric field yielding a maximal induced dipole moment.²⁹ After DEP-induced reorientation, the electric field initially intersects perpendicular to the particle surface. Consequently, ions in the liquid preferentially accumulate at the metallic hemisphere, yielding an induced charge cloud. During a steady state phase of an electric field application, the induced charge cloud expels the electrical field lines that are parallel to the particle surface. Consequently, fluid is drawn to the particle on a vector that is parallel with the electric field and then ejected away from the metallic hemisphere on a vector that is perpendicular to the electrical field.³⁰ This asymmetric fluid ejection results in particle propulsion in the direction of the dielectric hemisphere.

ICEP behavior of Janus particles has been reported using different metallic patch shapes and geometries.^{29, 31-36} Further, it has been reported that ICEP of nonfunctional Janus particles is useful for the transport of secondary functional cargo particles in biosensing applications.³⁷ This system was designed to sense numerous different biomolecules with the same Janus particle population. Yet, there are no reports demonstrating direct and specific biomolecule detection via ICEP with ICEMs.

As disclosed herein, ICEP offers a unique approach for direct, label-free detection of biomolecules. Indeed, it has been shown that induced electroosmotic flows at a flat metal surface, analogous to those seen in ICEP of a Janus particle, can be affected by surface contaminants. This arises from dielectric shielding of the polarizable surface as well as “buffering” of ions through surface reactions, which generally reduce the magnitude of the induced flows. 38

Such dielectric shielding of polarizable surfaces has been corroborated with Janus particles coated with poly(L-lysine)-g-poly(ethylene glycol) where the electrokinetic speed was suppressed as compared to that of uncoated particles due to shielding of surface charges.³⁹⁻⁴¹ Additionally, when protein was adsorbed on the gold hemisphere of gold-polystyrene Janus particles DEP similarly lead to dielectric shielding and a reduction in polarizability.⁴²

### B. Polystyrene Janus Particles

The most common Janus particles utilized in ICEP studies comprise a polymer (e.g., polystyrene (PS)) with a gold-layered hemisphere. A polymer particle is preferred as they are widely available and have dielectric properties. A gold patch (e.g., layer), is preferable because it has a significantly greater polarizability than most polymers.^{29,34,39,43} In one embodiment, the presently disclosed ICEMs comprise gold layered-PS Janus particles, wherein the PS is carboxylated to permit conjugation of aminated chemical compound species (e.g., polyethylene glycol-amine).

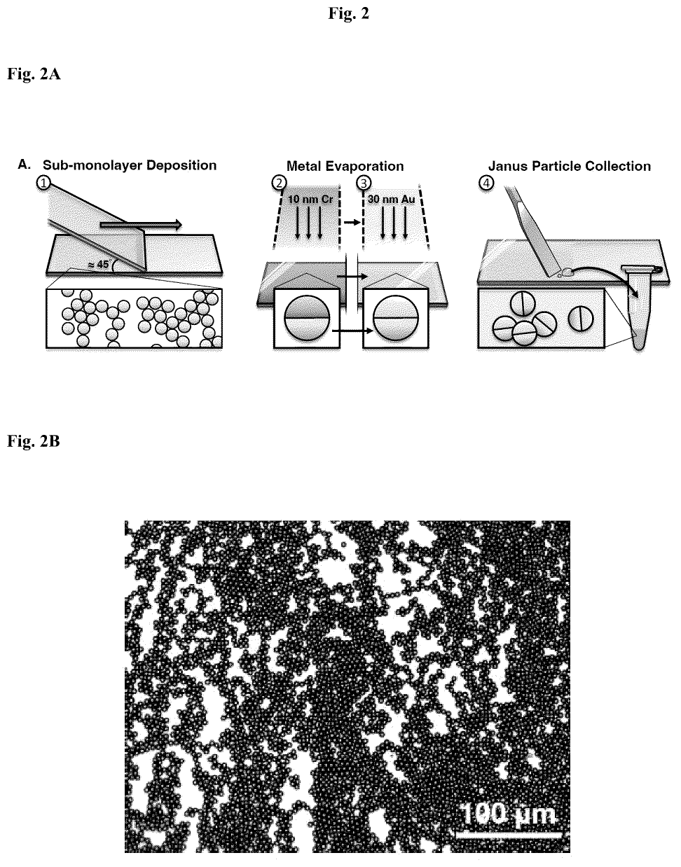

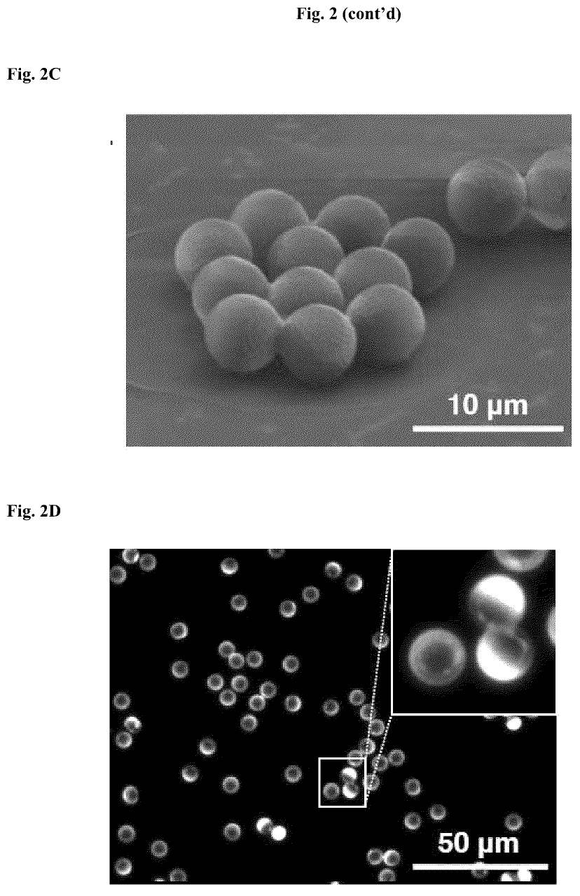

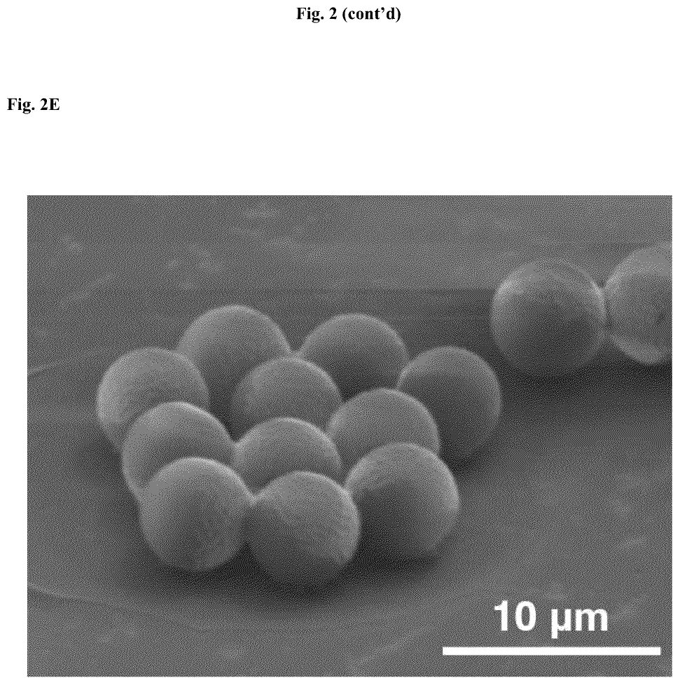

Unfunctionalized Janus particles were produced by a previously reported convective assembly-based approach.⁴⁴ In short, carboxylated, red fluorescent PS particles were deposited in a submonolayer on borosilicate glass slides to ensure that the particles did not overlap. See, FIG. 2A. Proper sub-monolayer deposition was confirmed via brightfield microscopy. See, FIG. 2B. Gold-layered PS Janus particles were then created by coating the unfunctionalized particles with sub-monolayers of chromium (˜10 nm) and gold (˜30 nm) using electron beam evaporation.

The gold-layered Janus particles were then collected and resuspended in pure milli-Q water (MQW). It is preferable not to resuspend in a high ion content solution as ICEP suppression has been reported under those conditions.²⁹ Scanning electron microscopy (SEM) and fluorescence microscopy confirmed that the metallic (e.g., gold) patch (e.g., layer) completely covered one hemisphere of the Janus particles and without any shape irregularities. See, FIGS. 2C and 2D. Although it is not necessary to understand the mechanism of an invention, it is believed that a high proportion of the created gold-layered PS Janus particles had metallic hemisphere layers without any irregularities.

Unfunctionalized Janus particles were then tested in an ICEP propulsion chamber. A coplanar electrode setup was typically used where two electrodes are positioned parallel on the same plane with a small gap separating them.^{29,34} Alternatively, a sandwich electrode setup may be used in which planar electrodes-often indium-tin oxide coated glass slides—are assembled on top of one another.^{37,45} During ICEP testing, metallodielectric particles travel perpendicular to the applied field. As such, in the sandwich setup, particles undergoing ICEP can travel in any direction parallel to the electrode planes (i.e., in the x- and y-direction), whereas in the coplanar setup, the particles only travel parallel to the electrode edges (i.e., in the x-direction). Thus, the coplanar electrode setup provides a more favorable environment for identifying particles undergoing true ICEP rather than other propulsion mechanisms, such as bulk fluid flow.

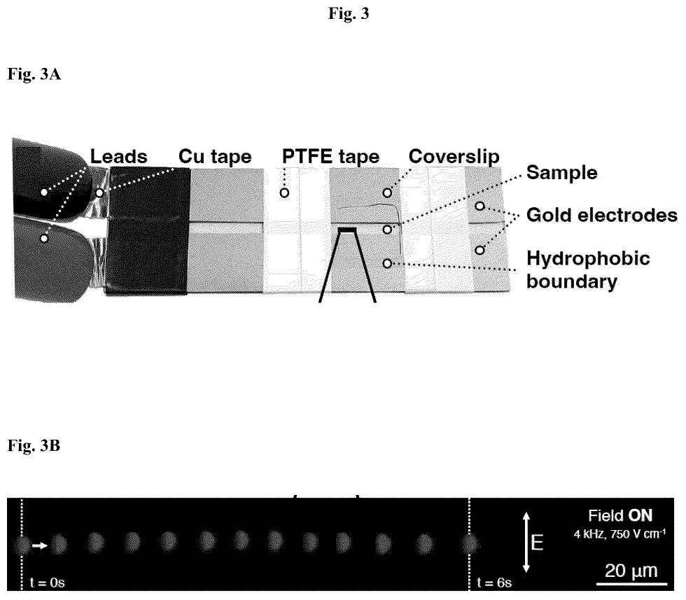

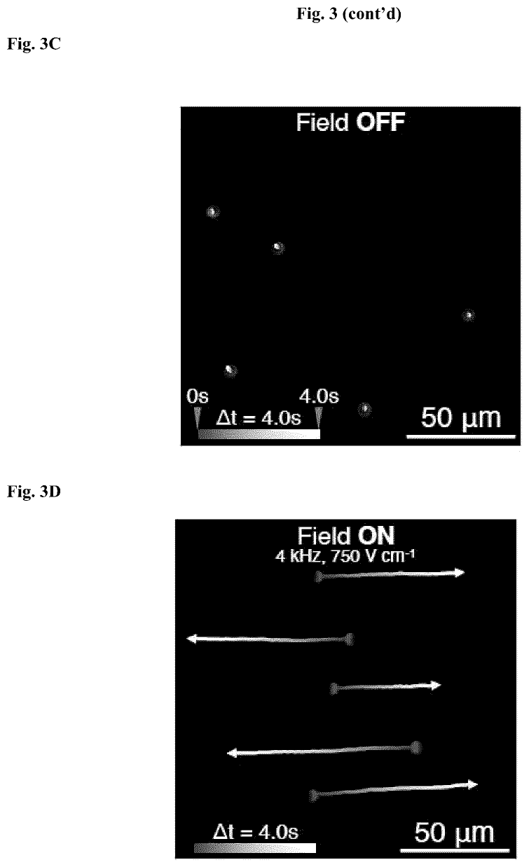

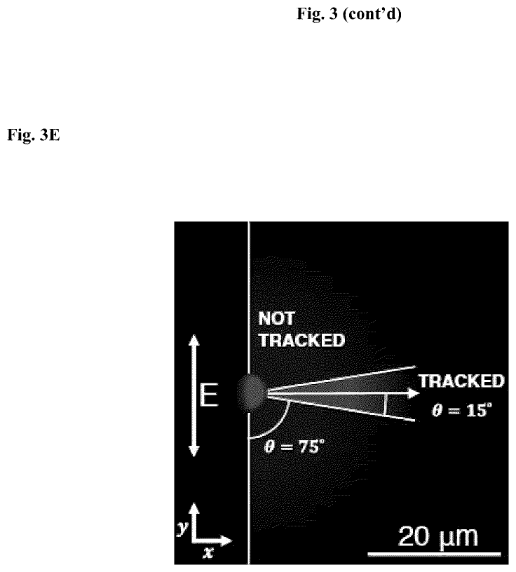

The data presented herein was collected using a coplanar electrode propulsion chamber setup. The electrodes were fabricated by depositing chromium (˜20 nm) and gold (˜100 nm) on standard borosilicate glass slides. The non-electrode slide surfaces were masked with a thin strip of Kapton tape spanning the length of the slide. After Cr/Au deposition, the mask was removed to reveal the two electrodes deposited on the unmasked areas. Leads were then attached to the electrode deposits using conductive copper tape. See, FIG. 3A. A small square outline overlapping the two Cr/Au electrodes was marked using a hydrophobic pen and two pieces of polytetrafluoroethylene (PTFE) tape were placed perpendicular to the electrodes at the boarder of the hydrophobic boundary. Finally, a particle aliquot (˜10-20 μL in MQW) was pipetted onto the propulsion chamber within the hydrophobic boundary and a coverslip was placed on top of the PTFE tape. A square wave (e.g., 4 kHz) was applied to the propulsion chamber to generate an AC electric field within the chamber (˜500-1000 Vcm⁻¹).

Chamber operation was evaluated by loading a solution of unfunctionalized Janus particles (˜2.5×10⁵ particles mL⁻¹) in MQW into a propulsion chamber situated on a fluorescence microscope equipped with a camera which video recorded particle movement during the application of a 4 kHz, 750 V cm⁻¹ electric field. The data showed that the majority of Janus particles reoriented and exhibited linear propulsion parallel to the chamber electrodes. Fiji/ImageJ® analysis software was used to analyze the captured video recordings and determine mean particle speeds in the x-direction. A representative time lapse photograph of a single particle undergoing ICEP shows motion that is clearly distinguishable from Brownian motion. FIG. 3B cf FIG. 3C. Further, multiple particles were tracked in a single recording. See, FIG. 3D.

Some particles did not travel parallel to the chamber electrodes, but instead were biased toward one electrode during their linear motion. Alternatively, occasional helical trajectories of particles were also observed in the chamber. Although it is not necessary to understand the mechanism of an invention, it is believed that these alternative modes of propulsion likely indicate that some particles had irregularities in overall shape and/or gold patch shape.⁴⁶ To eliminate data analysis bias induced by these irregularities, a strict tracking criterion was implemented to only analyze speeds of particles travelling within 15° of parallel to the electrode edges at the water-coverslip interface. See, FIG. 3E. This is because it has been reported that the tendency for particles to rise to the water-coverslip interface during reorientation arises at least partially due to DEP forces, but linear motion at the water-coverslip interface is due strictly to ICEP.²⁹

### C. Janus Particle Biomolecule Conjugates

In one embodiment, the present invention contemplates a composition comprising a functionalized Janus particles conjugated to a biomolecule capable of performing an ICEM assay. In one embodiment, the Janus particle is functionalized with a biomolecule recognition element.

When designing a biomolecule detection assay, one criterion is to minimize matrix effects, as these may interfere with the assay and lead to false results.⁴⁷ Although it is not necessary to understand the mechanism of an invention, it is believed that nonspecific adsorption of proteins or other molecules to either ICEM hemisphere potentially influences their IC electrophoretic motion. In one embodiment, an ICEM comprises a polyethylene glycol (PEG) antifouling layer on the PS hemisphere, as well as between the gold hemisphere and biotin recognition element.⁴⁸

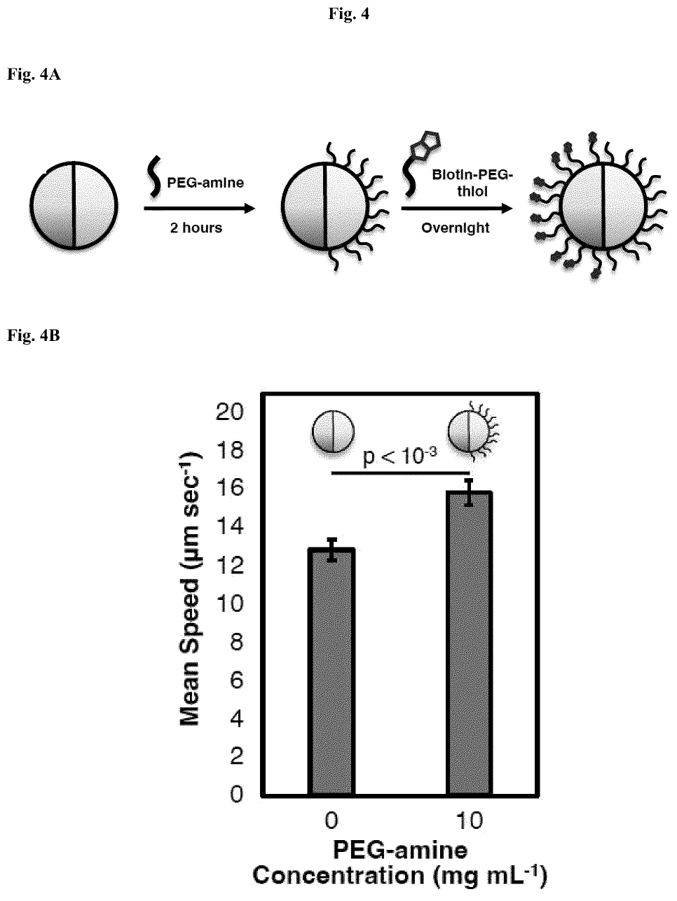

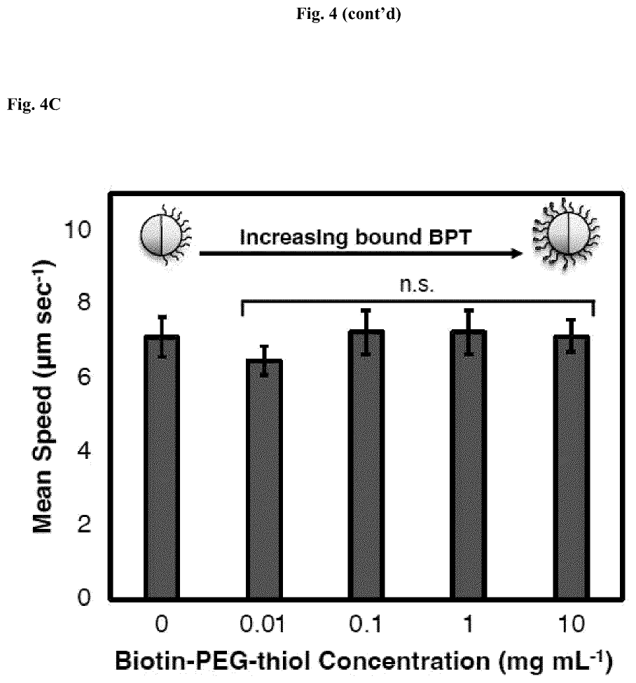

One type of antifouling layer was created by conjugating a PEG-amine (˜1 kDa) polymer to a Janus particle PS hemisphere using simple carbodiimide chemistry.⁴⁹ In short, Janus particles were mixed in a solution of 1-ethyl-3-(3-dimethylaminopropyl) carbodiimide (EDC) and 1 kDa mPEG-amine in 2-(N-morpholino) ethanesulfonic acid (MES) buffer for 2 hours at room temperature. See, FIG. 4A.

Because other PEG-containing molecules have been shown to suppress ICEP, the impact of a PEG-amine layer was assessed on ICEP to determine if this functionalization also suppressed particle motion.³⁹ For example, PEG-amine modified particles were washed with MQW and tracking experiments were performed to determine PEG-amine particle electrokinetic speed. The data showed that the addition of a PEG-amine layer on the polystyrene side of particles increased particle speed from 12.8 to 15.8 μm s⁻¹. See, FIG. 4B. Although it is not necessary to understand the mechanism of an invention, it is believed that this moderate increase in electrokinetic speed is likely not due to any changes to the asymmetry in polarizability of the particles, as the polymeric PEG-amine layer is not expected to dramatically lower the polarizability of the already weakly polarizable PS hemisphere. Instead, because the tracked PEG-amine particles were located at the water-coverslip interface, it is likely that this increase in speed is due to decreased interactions between the particles and the glass slide.⁵⁰

To functionalize ICEMs for specific capture of a biomolecule, biotin was selected as an exemplary recognition element to capture an exemplary biomolecule, streptavidin (SA). Biotin is a small molecule while streptavidin is a protein with a molecular weight of roughly 60 kDa,⁵¹ Both molecules are well known for use as model biomolecules.^{37,52}

Biotin recognition elements (biotin-PEG-thiol) with an integrated antifouling layer (PEG) were attached to the Janus particle gold-layered hemisphere with a terminal thiol group. Notably, it has been reported that this PEG linker conjugate allows a more favorable biotin orientation which enhances SA capture, relative to surface-bound biotin.⁵³ Biomolecule recognition element functionalization of the Janus particle gold hemisphere involved mixing PEG-modified particles in a solution of 1 kDa biotin-PEG-thiol (BPT) and 3 mM ethylenediaminetetraacetic acid (EDTA) in phosphate buffered saline (PBS) overnight. As modification of the particle gold hemispheres may be expected to lead to changes in the induced charge electrophoretic motion of

ICEMs, the particles were functionalized using a range of BPT concentrations and electrokinetic particle speed was assessed after washing into MQW. The data showed that the various increased concentrations of BPT did not lead to any significant changes in particle electrokinetic propulsion speed. FIG. 4C. Because no significant changes were observed between binding conditions, the 10 mg mL⁻¹ BPT concentration was selected as a standard functionalization condition to maximize the amount of BPT conjugated to the gold surface.

SA detection assays were then performed using the above-described biotin functionalized ICEM Janus particles. In short, ICEMs with PEG-functionalized PS hemispheres and PEG-biotin-functionalized Au hemispheres were prepared. After conjugation, the particles were washed four times into 1×PBS. Then, a 2.5×10⁵ ICEM aliquot was resuspended in a solution of

Alexa 594-conjugated SA in PBS (˜ 100 μL) including an SA concentration ranging between approximately 0-2×10⁴ nM. The mixtures were allowed to incubate while mixing at room temperature for 30 minutes. After incubation, the mixtures were washed in MWQ, placed in an electrokinetic propulsion chamber and relative electrokinetic speeds were determined. See, FIG. 5A.

The data showed that ICEM electrokinetic motion was substantially suppressed at all SA concentrations and was significantly different from control starting at 10 nM SA and above (P<0.05). See, FIG. 5B. Incremental SA concentration above 100 nM did not further suppress particle movement below a roughly 46% maximum reduction in speed. These data were fit to a 4-parameter logistic curve (4PL), commonly used to fit experimental data from biosensing assays such as ELISA.⁵⁴ Although it is not necessary to understand the mechanism of an invention, it is believed that ICEM assay sensitivity could be decreased simply and effectively by reducing the number of ICEMs incubated with the biomolecule sample; this would increase the ratio of target biomolecules to particles and likely manifest as a leftward shift of the concentration-ICEM speed response curve.

Representative particle tracks from both the control (0 nM SA) and the maximum SA concentration (2×10⁴ nM SA) demonstrate a clear visual difference between particle speed incubated with, or without, SA. See, FIG. 5C. Although it is not necessary to understand the mechanism of an invention, it is believed that modest variabilities in recorded speeds for a given SA condition may have originated from inhomogeneities in functionalization or differences in particle size or patch morphology not detected during studies, and minimization of these sources of deviation should be a target for future implementation of ICEMs.

To confirm that the observed decrease(s) in ICEM electrokinetic speed was due to SA-biotin capture rather than nonspecific adsorption, ICEMs were functionalized with preformed SA-biotin complexes (e.g., SA was blocked with a molar excess of free biotin). SA-biotin complexes were diluted to 1000 nM in PBS. Aliquots of 2.5×10⁵ functionalized particles were subjected to ICEM analysis under the following conditions: i) 1000 nM SA; ii) 1000 nM SA-biotin complex; and iii) 1000 nM free biotin. After incubation, particles were washed four times into MWQ, placed in the propulsion chamber, and tracked as described above.

The data showed that neither ICEMs incubated in free biotin nor ICEMs incubated in complexes of SA-biotin exhibited decreased speeds relative to the control, but that those incubated in 1000 nM SA exhibited a significant decrease in particle speed. See, FIG. 5D. These data indicate that the observed decrease in ICEM electromotive speed is, in fact, due to specific SA-biotin capture by particles via biotin-binding sites. It further provides evidence that the antifouling PEG layers may prevent nonspecific adsorption of protein to the gold hemisphere of the particles.

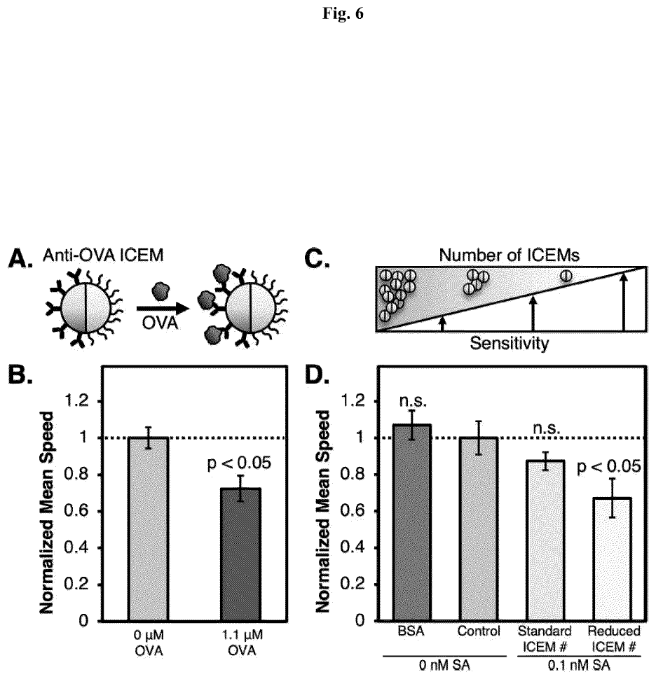

To evaluate the potential of the ICEMs to detect alternative target biomolecules, a protein OVA was tested using antibody recognition. To facilitate this assay, functionalized ICEMs with PEG on their PS hemispheres and anti-ovalbumin immunoglobulin G (anti-OVA IgG) on their gold hemispheres as recognition elements for OVA were prepared. See, FIG. 6A. Anti-OVA ICEMs (˜2.5×10⁵) were incubated with 0 and 1.1 μM OVA in PBS for 30 min while mixing at room temperature. After incubation, particles were washed and tracked as described above. The data show that anti-OVA ICEMs incubated with OVA exhibited a significant decrease in propulsion speed relative to the control without OVA. This result suggest that the use of antibodies as ICEM recognition elements substantially increases the breadth of potential detectible targets and thus the diagnostic value of ICEMs.

In addition to detecting other biomolecules, the sensitivity of the assay was improved by reducing the number of ICEMs per sample, therefore increasing the ratio of target biomarkers to particles. See, FIG. 6C. Assay performance was compared between a low SA concentration (i.e., 0.1 nM) using both the standard ICEM population (i.e., ˜2.5×10⁵) and an ICEM population that was reduced by 25-fold (i.e., ˜1×10⁴). In addition to evaluating the speed of ICEMs not incubated with SA, the speed of ICEMs incubated with 0.1 nM bovine serum albumin (BSA) was also measured as a control to assess specificity. The data showed that the speed of ICEMs incubated in SA at a standard ICEM number did not differ significantly from that of a control but that by using the reduced ICEM amount, significant decrease in ICEM speed was observed. See, FIG. 6D. Thus, the sensitivity of the assay can be conveniently tuned by adjusting the number of ICEMs. Furthermore, no significant decrease in speed was observed for ICEMs incubated with BSA. This provides further evidence that the antifouling layers, such as PEG, prevent nonspecific adsorption of proteins.

In summary, the above data suggests that ICEP of functionalized metallodielectric Janus particles (ICEMs) provide simple, label-free biosensing. For example, ICEP has been shown as a mechanism of direct signal transduction for biomolecule detection by determining respective electrokinetic speeds. ICEMs functionalized with an antifouling PEG layer and biotin recognition elements that capture SA showed that decreased ICEM speed occurred only in the presence of a SA-biotin complex. Moreover, a concentration-dependent decrease in speed was observed between 1-100 nM SA, likely due to increased dielectric shielding of the gold patch. Although it is not necessary to understand the mechanism of an invention, it is believed that this simple recognition element conjugation approach, functionalized ICEMs can now be tuned for recognition and capture of a range of biomolecules of interest by conjugating aptamers, antibodies, enzymes, or other recognition elements to the gold hemisphere of the particles.

### II. Shape-Encoded Electrokinetic Particles for Multiplexed Biosensing

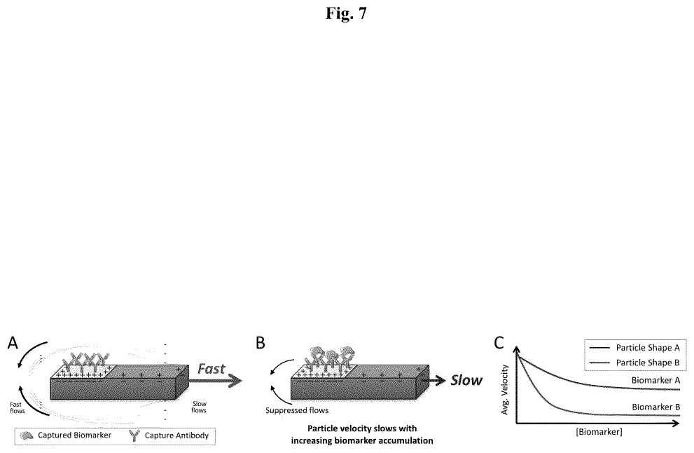

Understanding the factors that influence the motion of electrokinetic active particles enable the development of devices and methods to detect heterogeneous biomarkers. Electrokinetic active particles (EAP) are reported to have asymmetric surface polarizabilities and can be made by placing conductive patches on dielectric particles. Shields et al., “Supercolloidal Spinners: Complex Active Particles for Electrically Powered and Switchable Rotation” Advanced Functional Materials 2018, 28(35). For example, at high electric field frequencies, the conductive patches induce a charged screening cloud in the proximate fluid, driving nearby ions to flow more rapidly than ions near the dielectric regions. See, FIG. 7A. These EAPs subsequently undergo induced charge electrophoresis propulsion (ICEP), a phenomenon in which the speed of propulsion slows as the metal films lose their conductivity. Pascall et al., “Induced charge electro-osmosis over controllably contaminated electrodes” Phys Rev Lett 2010, 104(8), 088301. The capture of biomarkers (e.g., proteins) by immobilized affinity ligands on the metallic films alters the speed of propulsion, enabling biomarker quantification through basic microscopy and particle tracking. See, FIGS. 7B & 7C.

In particular, specific accumulation of biomarkers on conductive patches of the EAPs alters propulsion in a manner that enables a direct quantification of biomarker concentration using particle tracking. Although it is not necessary to understand the mechanism of an invention is it believed that some characteristics effecting EAP motion include, but are not limited to: a) biomarker parameters such as molecular size, surface charge and/or concentration; b) antifouling coatings; c) biomarker biorecognition motif coating density effects of captured ligands on LOD and dynamic range; and d) data multiplexing advantages.

### A. The Relationship Between Surface Interactions And ICEP

### 1. Biomarker Size, Charge Density and Concentration

The effect of biomarker accumulation on ICEP has been modeled using a Poisson-Boltzmann distribution, Gupta et al., “Ionic Layering and Overcharging in Electrical Double Layers in a Poisson-Boltzmann Model” Phys Rev Lett 2020, 125(18), 188004. This technique utilizes numerical predictions of particle speed as a function of biomarker size, charge, and concentration. Predictions from this model are compared to experimental results with osteoprotegerin (OPG), a common biomarker for breast cancer. Odén et al., “Plasma osteoprotegerin and breast cancer risk in BRCA1 and BRCA2 mutation carriers” Oncotarget 2016, 7(52), 86687-86694. Polyethylene glycol (PEG) is attached to the dielectric layer of the EAP as an antifouling layer and is conjugated to anti-OPG antibodies on the gold patches. Alternatively, thiolated antibodies, using 2-iminothiolane, can be conjugated directly to the gold films. Wang et al., “Comparison of four methods for the biofunctionalization of gold nanorods by the introduction of sulfhydryl groups to antibodies” Beilstein J Nanotechnol 2017, 8, 372-380.

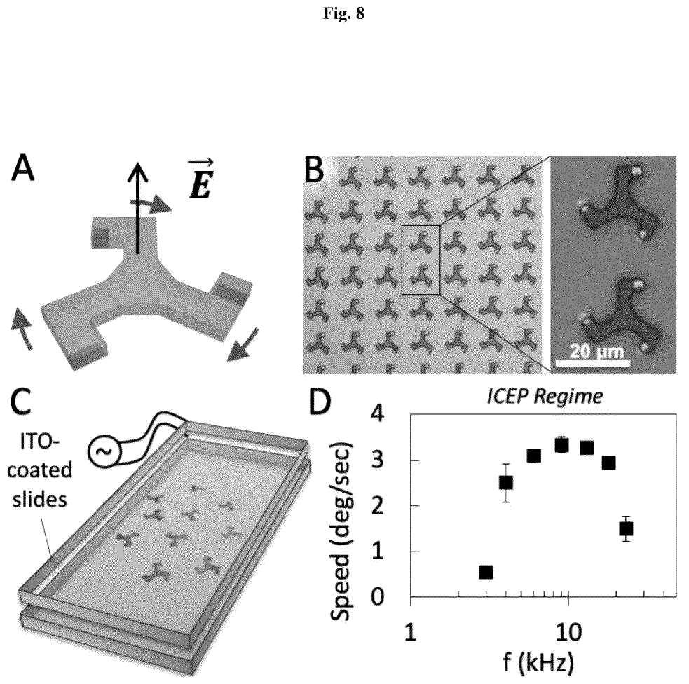

Due to the complex shape of EAPs and the discrete metal patches on their surfaces, the particles can harvest energy from alternating current (AC) electric fields and dissipate that energy through directed motions. See, FIG. 8. Owing to their flexible routes of fabrication, EAPs can be made in a variety of shapes with unique metallic coatings, allowing for precise control over particle motions outside of equilibrium. Particles driven at 3-50 kHz is used for impedimetric immunoassays because it relies on a mismatch in conductivity between adjacent surfaces. See Gangwal et al., “Induced-charge electrophoresis of metallodielectric particles” Phys Rev Lett 2008, 100(5), 058302; and FIG. 8D. Although it is not necessary to understand the mechanism of an invention, it is believed that a highly conductive surface adjacent to a dielectric surface gives rise to asymmetric fluid flows that drive particle motion (e.g., electrokinetic propulsion). As molecules (e.g., accumulate on the gold electrode, ion mobility is inhibited, slowing fluid flow in that region, and reducing particle speed.

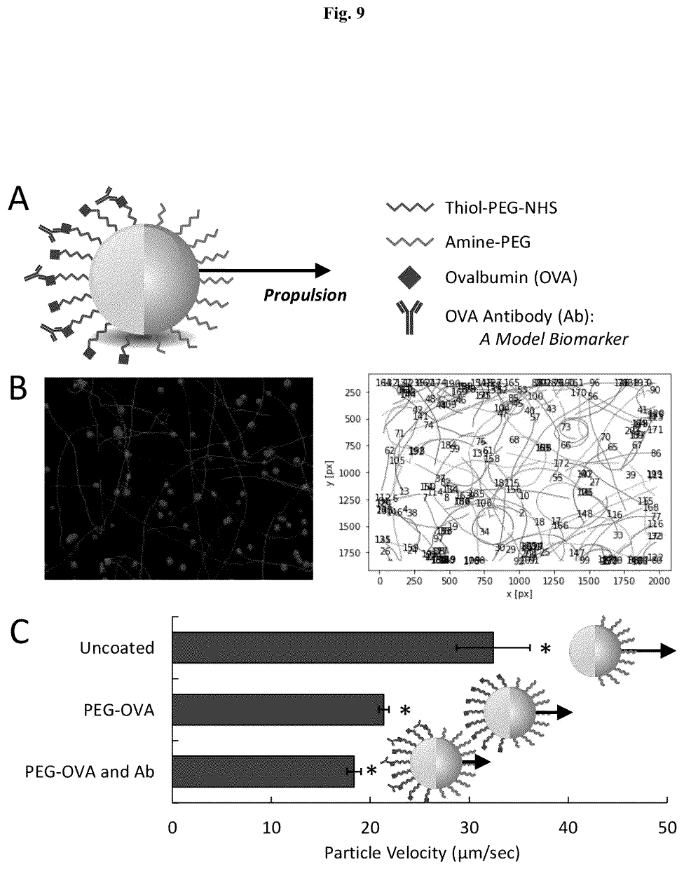

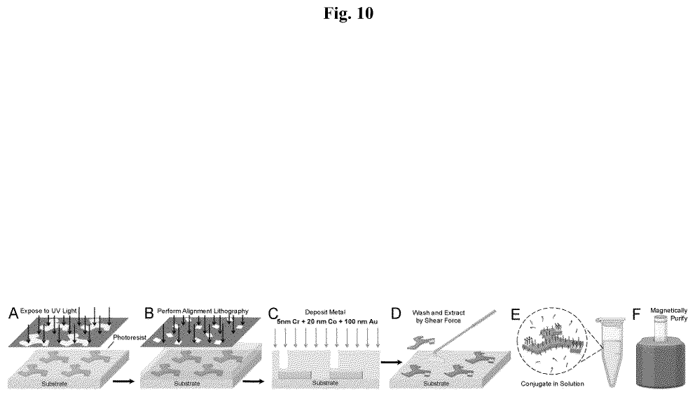

As shown above, particles can be driven across a range of field frequencies to undergo distinctive electrophoretic motions that guide their propulsion (e.g., speed). For example, Janus spheres were made by convective assembly followed by metal evaporation. The polymer side of the particles were coated with PEG using its amine-terminus to bind to a peptide carboxyl-terminus and the gold side of the particles were coated with a thiol-PEG N-hydroxysuccimide (NHS). See, FIG. 9A. Ovalbumin (OVA) was attached to the NHS groups via a reaction with primary amines. Using anti-OVA antibodies as a hypothetical biomarker, particle tracking was performed. See, FIG. 9B. Particle speed was measured, and it was observed that the particles did not travel in straight lines due to coating defects. See, FIG. 9C. Janus particles without attached OVA propelled the fastest (32.4±3.7 μm/sec) and particles with either attached OVA or OVA/anti-OVA complexes produced an average velocity of 21.3±0.5 and 18.4±0.7 μm/sec, respectively. This data suggests that the accumulation of biomarkers attached to EAP surface gold patches reduces particle speed in a manner that is readily detectable. In this way, the amount of material collected can be estimated by the altered fluid flow velocity resulting from changes in electrical impedance. Therefore, particles may be designed to capture biomarkers of interest for direct quantification by changes in particle speed. Such EAPs are fabricated, coated, and purified as shown. See, FIG. 10.

Although it is not necessary to understand the mechanism of an invention, it is believed that the interactions between: i) steric factors (e.g., EAP size and concentration); ii) electrostatic factors (e.g., EAP charge and surface pH); and iii) an antibody-biomarker complex and electrolytes, have a relationship with slip velocity of an electric double layer (EDL). For example, if biomarker size, charge, and concentration independently affect ICEP motion then these changes can be predicted using a modified Poisson-Boltzmann relationship.

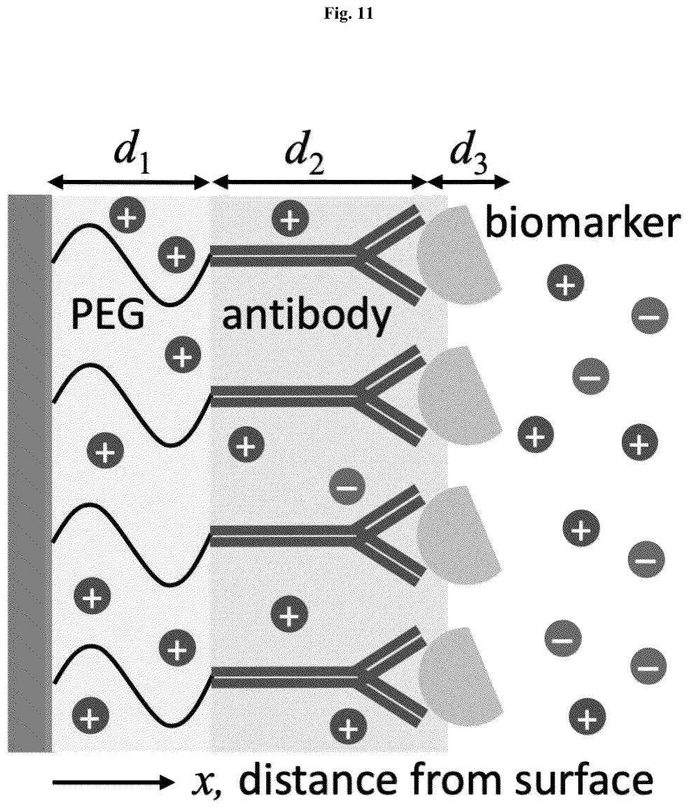

For simplicity, the complex geometry of electrokinetic particle motion can be approximated through a one-dimensional model. See, FIG. 11. The EAP surface comprises three layers: an antifouling layer (e.g., PEG), a biorecognition layer (e.g., antibodies), and a biomarker layer (e.g., OPG). Each layer is associated with: i) a characteristic thickness: d1, d2, and d3; and ii) a volumetric charge density associated with each layer: ρ1, ρ2, and ρ3. It is assumed that cations (e.g., H⁺) and anions (e.g., OH⁻) are mobile and follow a Boltzmann distribution. Kuron et al., “Moving charged particles in lattice Boltzmann-based electrokinetics” J Chem Phys 2016, 145(21), 214102.

A Poisson-Boltzmann equation models an electrical potential normal to the gold surface:

$${∇}^{2} ψ = \frac{{\partial }^{2} ψ}{\partial {x}^{2}} = - \frac{{ρ}_{1 , 2 , 3} + e ( {c}_{+} - {c}_{-} )}{{ε}_{r} {ε}_{0}} \qquad ( 1 )$$

where ψ is the electrical potential and εᵣ and ε₀ is the relative permittivity of the solvent and the permittivity of free space, respectively, and c_{±} are:

$${c}_{\pm } = {c}_{\pm 0} {e}^{∓ \frac{e ψ}{{k}_{B} T}} \qquad ( 2 )$$

where c_{±} and c_{±0} are the local and bulk concentration of ions. Different thicknesses of each layer as well as different charge densities can be evaluated. For example, by varying charge density, a direct way to study the effect of molecular charge density (i.e., by the zeta potential, ζ) and the packing density of molecules on the surface is provided. ρ_{1,2,3} is varied with the potential to include the effect of pH. A low potential regime model (i.e., e|ψ|<<k*T) extracts general scaling arguments and in a non-linear format (e.g., a high potential regime model) extracts quantitative or predictive relationships between biomarker size, charge density, pH, and concentration on propulsion speed.

This model can be validated, refined, and authenticated by study of sampling rate and population statistics on sensitivity. For example, detection of an OPG biomarker to diagnose cancer ranges between ˜120 to ˜40 ng/mL. As described above, anti-OPG antibodies can be bound to an EAP gold patch through thiol-PEG-NHS chemistry. Alonso et al., “Thiol-ene click chemistry towards easy microarraying of half-antibodies” Chem Commun (Camb) 2018, 54(48), 6144-6147.

Particle shapes that facilitate in situ rotation obviate the need for a motorized stage and enable the tracking of many particles within one field of view. Rotational speeds can be recorded using a conventional camera connected to an inverted microscope. Particle positions are then extracted frame-by-frame and assembled into spatiotemporal paths. Deterministic analyses connects the path data with a biomarker concentration obtained from calibration studies.

Other collected data evaluates variables including, but not limited to: i) image sampling rates (e.g., 1-50 Hz); ii) multiple electric field frequencies (e.g., 3-50 kHz); iii) multiple field strengths (e.g., 10-1,000 V/cm); iv) measured particle speed as compared to model predictions; and v) various solution pH. The concentration of EAPs may range between 10³-10⁶ particles/mL, where the definition for the limit of detection (LOD) is three standard deviations above baseline. Li et al., “Rapid capture of biomolecules from blood via stimuli-responsive elastomeric particles for acoustofluidic separation” Analyst 2021, 145(24), 8087-8096. These data elucidate the contribution of, or competition between, steric hindrance and electrolyte interactions on an EAP surface.

### 2. Electrically Permissive Antifouling Coatings

In one embodiment, the present invention contemplates an EAP comprising a surface with a gold patch, the gold patch coated with an electrically permissive antifouling film or layer. Although it is not necessary to understand the mechanism of an invention it is believed that an antifouling film modulates biomarker capture specificity.

For example, gold patches can be coated with polymer brushes (e.g., for example, oligoethylene glycol and sulfobetaine methacrylate). It has been reported that such polymer brushes reduce protein adsorption by minimizing protein unfolding. Faulón Marruecos et al., “Stabilization of Fibronectin by Random Copolymer Brushes Inhibits Macrophage Activation” ACS Applied Bio Materials 2019, 2(11), 4698-4702. Alternatively, gold patches are coated with a crosslinked porous matrix of bovine serum albumin (BSA), which reduces non-specific protein adsorption without affecting electrical permittivity. Sabate Del Rio et al., “An antifouling coating that enables affinity-based electrochemical biosensing in complex biological fluids” Nat Nanotechnol 2019, 14(12), 1143-1149. EAPs are also conjugated with an antibody directed against a biomarker (e.g., human epidermal growth factor receptor 2 (HER2)). Sauter, E. R., “Reliable Biomarkers to Identify New and Recurrent Cancer” Eur J Breast Health 2017, 13(4), 162-167. The LOD and specificity of the antifouling particles are compared to particles without antifouling coatings.

One example of how antifouling coatings improve sensitivity and specificity is that non-specific accumulation of plasma proteins can generate false signals. Antifouling coatings can reduce this non-specific adsorption and enhance specificity. In one embodiment, the present invention contemplates an EAP with a surface gold patch modified with an electrically permissive antifouling coating. In one embodiment, the antifouling coating is modified with capture antibodies. In one embodiment, the capture antibody has specificity against a protein biomarker. In one embodiment, the antifouling coating detects a biomarker with a similar LOD (e.g., <1 standard deviation) to EAPs without antifouling coatings. In one embodiment, the antifouling coating detects a biomarker with an improved LOD (e.g., <1 standard deviation) to EAPs without antifouling coatings.

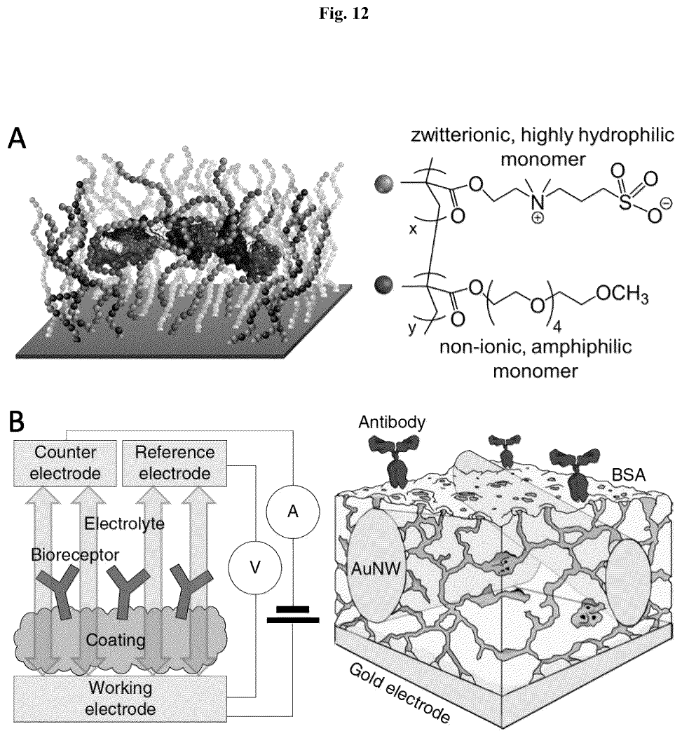

Numerous strategies have been attempted to affix antifouling coatings onto electrodes (e.g., uncrosslinked bovine serum albumin (BSA) and PEG); yet, these strategies have been shown to hinder electron transfer and mobility, thus reducing sensitivity. Banerjee et al., “Antifouling coatings: recent developments in the design of surfaces that prevent fouling by proteins, bacteria, and marine organisms” Adv Mater 2011, 23 (6), 690-718. Brushes, made from random copolymerization of hydrophilic zwitterionic monomers and nonionic amphiphilic monomers. can reduce protein fouling by preventing surface-induced protein unfolding. Faulón-Marruecos et al., “Stabilization of Fibronectin by Random Copolymer Brushes Inhibits Macrophage Activation” ACS Applied Bio Materials 2019, 2 (11), 4698-4702; See also FIG. 12A. Also, three-dimensional porous matrices of glutaraldehyde (GA)-crosslinked bovine serum albumin (BSA) can act as an antifouling surface, enabling high electrical sensitivities over long periods (i.e., 88% signal transmission after one month in plasma). Sabate Del Rio et al., “An antifouling coating that enables affinity-based electrochemical biosensing in complex biological fluids” Nat Nanotechnol 2019, 14(12), 1143-1149; and FIG. 12B.

### a. Copolymer Brush Antifouling Coatings

In one embodiment, the present invention contemplates an EAP with a surface gold patch attached to copolymer brushes comprised of oligoethylene glycol methacrylate (OEGMA) and sulfobetaine methacrylate (SBMA). In one embodiment, the copolymer brush is attached to the surface gold patch using atom transfer radical polymerization (ATRP). For example, the EAPs are first coated with trichloro[4-(chloromethyl)phenyl]silane, and the brushes are grown on the patches by adding SBMA and OEGMA (Mw=276 g/mol) at 100:0, 75:25, 50:50, 25:75, and 0:100 molar ratios of OEGMA to SBMA. To enable covalent biomarker antibody conjugation to the brush layer, methacrylic acid N-hydroxysuccinimide ester reacts with accessible primary amines on the antibodies.

Characterization of brush thickness and composition is performed by scanning electron microscopy (SEM) and Fourier Transform Infrared Spectroscopy-Attenuated Total Reflectance (FTIR-ATR). OEGMA/SBMA brushes made previously using this method had a dry thickness of 10 nm and water contact angles that decrease from 60° to 10° with increasing SBMA (data not shown). FTIR confirmed that the composition of the brushes matched that of the feed of the reaction.

EAPs attached to the antifouling brushes are measured for their ability to suppress electrokinetic non-specific protein adsorption by incubation in blood for 120 min. Particle speed is compared with controls to evaluate reductions in non-specific protein accumulation. Useful antifouling coatings have a high stability, low attenuation of propulsion, low protein adsorption and a clinically relevant specificity (e.g., HER2 captures at or below normal blood levels of ≤12 μg/mL). Antibodies can be added directly to the gold surfaces prior to brush polymerization or conjugated to the brush distal ends after polymerization. Weltz et al., “Dramatic Increase in Catalytic Performance of Immobilized Lipases by Their Stabilization on Polymer Brush Supports” ACS Catalysis 2019, 9 (6), 4992-5001.

Alternatively, PNIPAAm brushes may be used as an antifouling layer. Biomarker antibodies are bound to the distal ends of the thermally responsive PNIPAAm brushes, and non-specifically adsorbed proteins will be removed with cyclic heating. Ashaduzzaman et al., “Studies on an on/off-switchable immunosensor for troponin T” Biosens Bioelectron 2015, 73, 100-107.

### b. Glutaraldehyde/Bovine Serum Albumin (GA/BSA) Antifouling Coatings

Due to the porous network structure of the GA/BSA coatings, gold nanoparticles (NPs) can be added to enhance conductivity. See, FIG. 12B. Several parameters may be varied to produce uniform and stable antifouling coatings: i) BSA concentration; ii) GA concentration; iii) gold nanoparticle size; and iv) gold nanoparticle concentration. GA/BSA antifouling coatings can be prepared by a variety of methods, including but not limited to, mixing reagents, sonicating, centrifuging, and drop-casting.

GA/BSA antifouling coatings are usually deposited onto substrates containing the EAPs directly after fabrication. For drop-casted gold surfaces, biomarker antibodies are added either during EAP fabrication by interspersing between films or conjugated to the tops of the gold films via carbodiimide crosslinker chemistry. SEM can then determine film thickness and stability immediately after coating and after extraction from the wafer.

As described above, the GA/BSA EAPs can be tested for non-specific protein adsorption suppression. Particles will be calibrated to correlate particle velocity with HER2 concentration. Specificity of EAPs, including control EAPs (e.g., without an antifouling coating) can be determined by a double-blind study comprising 10 samples of normal and abnormal levels of HER2. Results will be compared to predicate HER2 biosensors, which display an average specificity ≥95% with a LOD at or below 12 ng/ml. Dekker et al., “Determining sensitivity and specificity of HER2 testing in breast cancer using a tissue micro-array approach” Breast Cancer Res 2012, 14 (3), R93.

### B. Electrokinetic Biosensing

### 1. Conventional Biotransduction Paradigms

Early biomarker detection has been best achieved with minimally invasive strategies through liquid biopsies. For example, Western blotting and enzyme-linked immunosorbent assays (ELISAs) remain the state of the art for biological detection in liquid biopsies in both research and clinical settings. Western blots separate proteins by molecular weight; however, this technique suffers from low throughput and complex workflows. Traditional ELISAs employ immobilized antibodies raised in animals directed against specific biomarkers. Once biomarkers are captured and purified, a secondary antibody with an enzyme is used to generate a visible color change that is proportional to the amount of biomarker captured. Due to the required two rounds of selectivity, ELISA is far more quantitative (e.g., 0.01-100 pg/mL limit of detection (LOD) than Western blotting. Nimse et al., “Biomarker detection technologies and future directions” Analyst 2016, 141(3), 740-55; and Zhang et al., “Predicting detection limits of enzyme linked immunosorbent assay (ELISA) and bioanalytical techniques in general” Analyst 2014, 139(2), 439-45. However, most ELISAs are designed around a single biomarker which requires numerous separately packaged reagents and 5 hours to complete on average. Hosseini et al., “Advantages, Disadvantages and Modifications of Conventional ELISA”. 2018; p 67-115.

In recent years, several alternative types of biotransducers have been proposed. Lee et al., “Microarray methods for protein biomarker detection” Analyst 2008, 133(8), 975-83; see also Table 1.

TABLE 1
Predicate biotransduction mechanisms.

| Category | Mechanism |
|---|---|
| Electrochemical | Produces or sequesters electrons. |
| | Cho et al., “Electrochemical biosensors: perspective on functional |
| | nanomaterials for on-site analysis” Biomater Res 2020, 24, 6. |
| Optical | Uses photons to inspect analytes. |
| | Damborsky et al., “Optical biosensors” Essays Biochem 2016, 60(1), 91- |
| | 100. |
| Electronic | Detects changes in surface charge. |
| | Syu et al., “Review-Field-Effect Transistor Biosensing: Devices and |
| | Clinical Applications” ECS Journal of Solid State Science and Technology |
| | 2018, 7 (7), Q3196-Q3207. |
| Gravimetric | Detects changes in mass. |
| | “Cali et al., “Gravimetric biosensors” Methods Enzymol 2020, 642, 435- |
| | 468. |
| Pyroelectric | Produces current from temperature changes. |
| | Naresh et al., “A Review on Biosensors and Recent Development of |
| | Nanostructured Materials-Enabled Biosensors” Sensors (Basel) 2021, 21(4). |
| Magnetic | Detects magnetic labels. |
| | Nabaei et al., “Magnetic biosensors: Modelling and simulation” Biosens |
| | Bioelectron 2018, 103, 69-86. |

These technologies exploit numerous types of physicochemical transduction to convert the presence of biomarkers to a useful readout (e.g., optical or electrical signal). This includes surface-enhanced Raman spectroscopy (SERS), which leverages the plasmonic effect for quantification, surface plasmon resonance (SPR), quartz crystal microbalance (QCM), and mass sensing BioCD protein arrays. Smolsky et al., “Surface-Enhanced Raman Scattering-Based Immunoassay Technologies for Detection of Disease Biomarkers” Biosensors (Basel) 2017, 7(1); Bellassai et al., “Surface Plasmon Resonance for BiomarkerDetection: Advances in Non-invasive Cancer Diagnosis” Front Chem 2019, 7, 570; Atay et al., “Quartz crystal microbalance based biosensors for detecting highly metastatic breast cancer cells via their transferrin receptors” Analytical Methods 2016, 8(1), 153-161; and Wang, X.; Zhao, M.; Nolte, D. D., “Prostate-specific antigen immunoassays on the BioCD” Anal Bioanal Chem 2009, 393(4), 1151-6. However, these techniques have failed to supplant traditional ELISA due to their limited selectivity or narrowly focused designs that limit the diversity of biomarkers that can be analyzed. Rusling et al., “Measurement of biomarker proteins for point-of-care early detection and monitoring of cancer” Analyst 2010, 135(10), 2496-511. Thus, there remains a broad need for an in vitro biosensor that preserves the efficacy of ELISA, but offers improved workability and/or versatility.

Several factors drive an exigent need for improved biosensors. For example, success of therapeutic interventions is inextricably linked to the time of initial diagnosis. For pathologies beginning at the cellular or molecular level, early identification of rare disease-associated biomarkers—especially before symptoms manifest—can dramatically improve the likelihood of survival. Reliance upon a narrow set of biomarkers can greatly limit the accuracy of initial diagnoses, especially for heterogeneous diseases like cancer, which are associated with a range of biomarker morphologies, including antigens, cell free DNA (cfDNA), exosomes, and circulating tumor cells (CTCs). Weigel et al., “Current and emerging biomarkers in breast cancer: prognosis and prediction” Endocr Relat Cancer 2010, 17(4), R245-62. For pathologies that progress rapidly (e.g., hours for many infectious diseases), slow readouts can harm outcomes and increase the cost of intervention. Thus, there remains a need to study new biosensing approaches to address these challenges.

### 2. Electrokinetic Active Particle Biosensors

Given the above discussed outstanding challenges of the state of biosensor art, the present invention provides the necessary improvements for a next-generation biosensor including, but not limited to:

### Faster Speed

Traditional ELISAs take 5 hours to perform, on average. While faster workflows may not change the course of therapeutic intervention for some diseases (e.g., cancer, where timescales of treatment are on the order of weeks), a faster approach can have a higher likelihood for adoption and impact other areas of need where rapid detections are essential (e.g., HSV-1/2, varicella zoster virus, enterovirus, parechovirus, influenza, bacterial resistance, and SARS-COV-2). Caliendo et al., “Better tests, better care: improved diagnostics for infectious diseases” Clin Infect Dis 2013, 57 Suppl 3, S139-70.

### Higher Sensitivity

Poor patient outcomes and high cost are two drivers behind the urgent need for new biosensing strategies with improved sensitivity. As diseases progress, the success of therapeutic interventions typically decreases, and their associated cost increases. DeSantis et al., “Breast cancer statistics, 2017, racial disparity in mortality by state” CA Cancer J Clin 2017, 67 (6), 439-448. Thus, there is a need for biosensing strategies that offer higher sensitivity to enable the detection of rare disease-associated biomarkers earlier in the courses of disease progression.

### Increased Versatility

Many diseases are highly heterogeneous, producing numerous types of biomarkers. Thus, there is a need for technologies that cut across biospecimen types and enable multimodal readouts. A biosensing strategy capable of detecting proteins, nucleic acids, and rare cells simultaneously would elevate the impact of liquid biopsy-based biosensors that cut across disease types. For instance, only about 25% of breast cancers are HER2+. Thus, systems focused solely on HER2 detection leave a majority of the tested breast cancer population with false negatives. Sauter, E. R., “Reliable Biomarkers to Identify New and Recurrent Cancer” Eur J Breast Health 2017, 13 (4), 162-167. Technologies that distinguish and quantify multiple types of biomarkers are poised to have a greater impact in real-world testing scenarios.

In one embodiment, the present invention contemplates a liquid biopsy-based biosensor that integrates electrokinetics, biointerfaces, and soft matter physics. For example, when stimulated by AC electric fields, the gold patches on the EAPs induce a charged screening cloud in the adjacent fluid, driving nearby ions to flow more rapidly than ions over the dielectric regions which results in an asymmetric fluid flow around the particle. In one embodiment, biorecognition motifs are appended to the gold patches such that biomarkers (e.g., proteins) accumulate on the gold patch, thereby altering the electrical permittivity of the particle surface. This alteration in electrical permittivity affects the speed of propulsion in a manner that corresponds to the amount of accumulated material. Thus, the mechanism of detection is built on readily observable changes in particle speed.

EAP-based biosensors has advantages over other conventional biosensing technologies by its ability to detect biomarkers at a single-particle level. By decoupling the procedures of detection from the physical hardware (e.g., the plate, gel, microfluidic device etc.), these presently disclosed electrokinetic particles can detect biomarkers in a smaller and yet parallel manner. By altering particle shape, specific biomarkers of interest can be identified and quantified so that several different biomarkers can be simultaneously detected and distinguished in a single assay. Nimse et al., “Biomarker detection technologies and future directions” Analyst 2016, 141(3), 740-55. Since the geometry of the gold patches can also be tailored, the EAP biosensors can capture and detect several types of biomarkers (e.g., proteins, nucleic acids, exosomes, and cells) simultaneously.

Although it is not necessary to understand the mechanism of an invention, it is believed that the presently disclosed EAP biosensors have distinct advantages over conventional biosensors by uniquely combining electrokinetic active particles with an impedance-based detection device. EAP biosensors detect an array of heterogeneous biomarkers simultaneously, thereby providing a significant advance over traditional ELISA methods, especially for pathologies that cannot be narrowly defined by a single biomarker. For example, conventional electrochemical impedance assays (EIAs) use biorecognition motifs, usually antibodies, immobilized on conductive substrates to measure small changes in conductivity as biomarkers accumulate. Bahadir et al., “A review on impedimetric biosensors” Artif Cells Nanomed Biotechnol 2016, 44 (1), 248-62; and Bertok et al., “Electrochemical Impedance Spectroscopy Based Biosensors: Mechanistic Principles, Analytical Examples and Challenges towards Commercialization for Assays of Protein Cancer Biomarkers” ChemElectroChem 2018, 6(4), 989-1003. For example, it has been reported that EIAs can achieve a LOD below 1 μg/mL (i.e., 2, 40, 200, and 280 fg/mL) for HER3, which is below the cutoff in normal serum. Hou et al., “DNAzyme-functionalized gold-palladium hybrid nanostructures for triple signal amplification of impedimetric immunosensor” Biosens Bioelectron 2014, 54, 365-71; Sonuc et al., “Ultrasensitive electrochemical detection of cancer associated biomarker HER3 based on anti-HER3 biosensor” Talanta 2014, 120, 355-61; Canbaz et al., “Fabrication of a highly sensitive disposable immunosensor based on indium tin oxide substrates for cancer biomarker detection” Anal Biochem 2014, 446, 9-18; Canbaz et al., “Electrochemical biosensor based on self-assembled monolayers modified with gold nanoparticles for detection of HER-3” Anal Chim Acta 2014, 814, 31-8; and Asav et al., “A novel impedimetric disposable immunosensor for rapid detection of a potential cancer biomarker” Int J Biol Macromol 2014, 66, 273-80. However, most EIAs are cumbersome to operate or require complex microfluidic setups. Shamsi et al., “A digital microfluidic electrochemical immunoassay” Lab Chip 2014, 14 (3), 547-54.

### 3. Antibody Coating Density Modulation of EAP Biosensor Sensitivity

In one embodiment, the present invention contemplates a first electrokinetic active particle (EAP) with a first antibody coating density and a second electrokinetic active particle with a second antibody coating density, wherein said second EAP has a lower antibody coating density than the first EAP antibody coating density. Although it is not necessary to understand the mechanism of an invention, it is believed that an EAP LOD and dynamic range is modulated by the surface antibody coating density. It is further believed that a lower antibody coating density provides a lower LOD and a lower dynamic range. For example, EAP gold patches can be coated with different antibody densities (e.g., OPG-antibodies or HER2-antibodies) with solutions comprising different antibody concentrations that correlate with their respective in vivo concentrations. For example, a conventional ELISA has an 8-fold lower sensitivity toward HER2 (185 kDa) than OPG (60 kDA) because in vivo it is a less abundant protein (e.g., 8 μg/mL vs. 1 μg/mL, respectively). Sauter, E. R., “Reliable Biomarkers to Identify New and Recurrent Cancer” Eur J Breast Health 2017, 13 (4), 162-167; and Asgeirsson et al., “Serum epidermal growth factor receptor and HER2 expression in primary and metastatic breast cancer patients” Breast Cancer Research 2007, 9 (6), R75.

### 4. Multiplexed Detection of Diverse Biomarkers

In one embodiment, the present invention contemplates a composition comprising a plurality of EAPs wherein each of the plurality of EAPs is attached to a biomarker antibody. In one embodiment, the biomarker antibody is specific for a biomarker including, but not limited to, OPG, HER2, IL-6, cfDNA, CD44⁺ exosomes, and CTCs. In one embodiment, the shape of a first EAP is different than the shape of a second EAP. In one embodiment, the first EAP is attached to a first biomarker antibody and the second EAP is attached to a second biomarker antibody. In one embodiment, the first biomarker antibody is specific for a different biomarker than the second biomarker antibody. In one embodiment, the first biomarker has a different coating density than the second biomarker antibody. In one embodiment, the shape of a first EAP is different than the shape of a second EAP. In one embodiment, the first EAP is attached to a first biomarker antibody and the second EAP is attached to a second biomarker antibody. In one embodiment, the first biomarker antibody is specific for a different biomarker than the second biomarker antibody. In one embodiment, the first biomarker has a different coating density than the second biomarker antibody.

Although it is not necessary to understand the mechanism of an invention, it is believed that shape-encoding of EAPs provide for the simultaneous detection of multiple diverse types of biomarkers and broaden their dynamic range. It is further believed that a multiplex EAP system accurately detects multiple biomarkers and thereby improve clinical diagnostic reliability. In particular, the multiplexed EAP system simultaneously detects different biomarkers including, but not limited to, proteins, nucleic acids, exosomes and/or whole cells. For each biomarker type, EAPs are modified (e.g., attached) with antibodies against a specific biomarker. Basal concentrations of the biomarkers are determined using conventional ELISA kits such that the LOD and dynamic range are compared and validated for each particle type via a series of calibration studies.

For example, exosomes are now recognized as a useful diagnostic tool. Exosomes are typically 30-150 nm in size and are secreted by most cells. Evidence shows that exosomes contain enriched sets of biomolecules from the cells. Zhang et al, “Exosomes: biogenesis, biologic function and clinical potential” Cell Biosci 2019, 9, 19. Tumor-derived exosomes possess proteins, RNAs, and other factors that constitute a biomolecular signature of the originating tumor. For example, CD44⁺ exosomes may provide a diagnostic biomarker for breast cancer, as neoplastic breast cancer cells have been reported to overexpress this protein. Jia et al., “Exosome: emerging biomarker in breast cancer” Oncotarget 2017, 8(25), 41717-41733; and Santos et al., “Exosome-mediated breast cancer chemoresistance via miR-155 transfer” Sci Rep 2018, 8 (1), 829. To make CD44+ exosomes, disseminated BT-20 cancer cells are isolated by ultracentrifugation. Shirure et al., “CD44 variant isoforms expressed by breast cancer cells are functional E-selectin ligands under flow conditions” Am J Physiol Cell Physiol 2015, 308(1), C68-78; and Wang et al., “Exosomes from M1-Polarized Macrophages Enhance Paclitaxel Antitumor Activity by Activating Macrophages-Mediated Inflammation” Theranostics 2019, 9(6), 1714-1727. Exosome size and concentration will be measured by SEM and DLS. Wu et al., “Exosomes: improved methods to characterize their morphology, RNA content, and surface protein biomarkers” Analyst 2015, 140 (19), 6631-42.

In addition, increased levels of cell-free DNA (cfDNA) in blood of cancer patients has been observed to correlate with shorter progression-free survival, refractory responses to treatment, and death. For example, cfDNA encoding KLK10, SOX17, WNT5A, and MSH2 all display higher degrees of methylation in patients with cancer than healthy patients. Panagopoulou et al., “Circulating cell-free DNA in breast cancer: size profiling, levels, and methylation patterns lead to prognostic and predictive classifiers” Oncogene 2019, 38(18), 3387-3401. Thus, methylated cfDNA is believed to serve as a prognostic indicator for some types of cancer. An exemplary antibody that binds to methylated DNA includes, but is not limited to an anti-acetylated H3 (K9/K14) antibody (EpiGentek).

More conventional diagnostic biomarkers may also be employed in the present multiplex EAP system. Human IL-6 (20.9 kDa) is useful because IL-6 blood levels are known to be ˜1.5 pg/mL in healthy patients versus ˜25.0 pg/mL in metastatic cancer patients. Ahmed et al., “Prognostic value of serum level of interleukin-6 and interleukin-8 in metastatic breast cancer patients” Egypt J Immunology 2006, 13(2), 61-68. Alternatively, circulating tumor cells (CTCs) can be captured by EpCAM which is abundantly expressed on BT-20 cells. Shields et al., “Magnetic separation of acoustically focused cancer cells from blood for magnetographic templating and analysis” Lab Chip 2016, 16(19), 3833-3844; and Sterzynska et al, “Analysis of the specificity and selectivity of anti-EpCAM antibodies in breast cancer cell lines” Folia Histochemica et Cytobiologica 2012, 50(4), 534-541. Because CTCs are extremely rare, often with concentrations as low as 1 cell in 10 mL of blood and that EAPs attached to CTCs will likely not undergo ICEP, a nuclear stain in combination with anti-CD45 antibodies will validate the EAP data regarding these biomarkers enumerating CTCs.

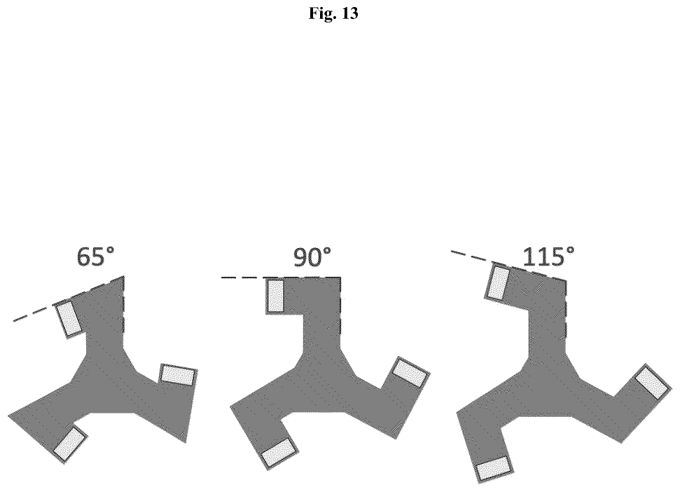

As each biomarker may be attached to a uniquely shaped EAP, optical microscopy provides a visual capability to distinguish between different biomarkers. See, FIG. 13. A panel of unique particle shapes is shown wherein each EAP has a different angular structure. For example, each unique EAP shape may be attached to a different biomarker, including but not limited to: i) HER2 consisting of 65° angular structures; ii) OPG consisting of 90° angular structures; and iii) a cfDNA consisting of 115° angular structure.

Alternatively, different EAP shapes may be distinguished by incorporation of fluorophores or aptamers using conventional techniques. Shields et al., “Field-directed of patchy anisotropic microparticles with defined shape” Soft Matter 2013, 9(38), 9219-29.

### III. Purification/Identification/Quantification Instrumentation

### A. Fourier Transform Infrared Spectroscopy-Attenuated Total Reflectance

Attenuated total reflectance (ATR) is the most widely used sampling methodology for Fourier transform infrared (FTIR) spectroscopy. ATR-FTIR quickly and easily measures a broad range of sample types, including liquids, solids, powders, semisolids, and pastes.

In ATR sampling, the infrared (IR) light travels through a crystal, is totally internally reflected at least once at the crystal-sample interface; and the reflected light travels to the FTIR detector. During the internal reflection, a part of the IR light travels into the sample, where it can be absorbed. This is called the evanescent wave. The penetration depth of the evanescent wave into the sample is defined by the refractive index difference between the sample and the ATR crystal. To account for different sample types and different pathlength requirements, several materials with different refractive indices are used as ATR sensors. For liquids and pastes, a small drop of the sample is placed onto the ATR crystal. The measurement is taken, and, after completion, the crystal can be wiped clean using a light solvent, if necessary. For powders, thin films, or other solid samples, the sample is placed onto the ATR crystal and pressed down using the swivel press to ensure optimal contact between sample and crystal. After the measurement, the sample can then be recollected, ideal for low-volume or expensive samples. The crystal can then be wiped clean using a light solvent, if necessary.

Most FTIR spectrometers come equipped with an ATR sampling station and Agilent benchtop and compact & portable FTIR spectrometers all offer ATR capabilities. The Agilent Cary 630 FTIR spectrometer accommodates a wide selection of ATR sensors and features the ability to switch from one ATR sensor to another instantaneously.

For most applications, single reflection zinc selenide (ZnSe), diamond, and germanium (Ge) ATR sampling modules are commercially available. These modules are used with a sampling press, and are excellent for analyzing solid materials, as well as liquids, pastes, and gels.

Single reflection measurements reflect only once within the ATR crystal, whereas multi-reflection ATR sensors use a longer crystal for multiple reflections. This creates a longer effective pathlength for increased sensitivity-ideal for challenging applications that require a lower limit of detection and faster data collection.

### B. Impedance-Based Spectroscopy

Electrochemical impedance spectroscopy (EIS) is a technique used for the analysis of interfacial properties related to bio-recognition events occurring at the electrode surface, such as antibody-antigen recognition, substrate enzyme interaction, or whole cell capturing. Thus, EIS could be exploited in several important biomedical diagnosis and environmental applications. However, the EIS is one of the most complex electrochemical methods, therefore, this review introduced the basic concepts and the theoretical background of the impedimetric technique along with the state of the art of the impedimetric biosensors and the impact of nanomaterials on the EIS performance. The use of nanomaterials such as nanoparticles, nanotubes, nanowires, and nanocomposites provided catalytic activity, enhanced sensing elements immobilization, promoted faster electron transfer, and increased reliability and accuracy of the reported EIS sensors. Thus, the EIS was used for the effective quantitative and qualitative detections of pathogens, DNA, cancer-associated biomarkers, etc. Magar et al., “Electrochemical Impedance Spectroscopy (EIS): Principles, Construction, and Biosensing Applications” Sensors (Basel). 2021 October; 21(19): 6578.

### 1. Potentiometric Analysis

A reference electrode and an indicator electrode are allocated in a simple electrochemical cell whereas the difference of potential between the two electrodes is recorded to provide significant information about the sample concentration. In the potentiometric technique, at zero current, the potential changes (vs. a reference electrode) are correlated to the changes of a concentration of a target analyte. The EMF of a cell depends on that concentration.

### 2. Coulometric Analysis

Coulometry is a method to carry out exhaustive electrolysis of an analyte by applying constant potential onto a working electrode surface with respect to a reference electrode.

Coulometric titrations are common practices to measure the sample. However, the constant-potential coulometry is not subjected to the effects of interferences since the potential of the working electrode is controlled at a value at which only a single electrochemical reaction is conducted.

### 3. Voltammetric Analysis

The sample is subjected to a constant/varying potential at the electrode's surface to record the Faradaic current produced. This technique is very important to understand the mechanisms and the kinetics of oxidation-reduction reactions and the electrochemical reactivity of an analyte. The voltammetry falls into two sub-classes termed as polarography and amperometry. Polarography is a voltammetric technique in which chemical species (ions or molecules) undergo oxidation or reduction at the surface of a polarized dropping mercury electrode (DME) at an applied fixed potential vs. a reference electrode. From the resulting current-voltage (I-V) curve, both the concentration and the nature of the oxidized and/or the reduced substance(s) adsorbed at the dropping mercury electrode surface could be determined. In amperometric methods, redox reactions (oxidation or reduction) of electroactive molecule(s) are measured at a constant potential. Application of voltammetry is widely exploited in biomedical diagnosis and environmental analysis.

### 4. Electrochemical Impedance Spectroscopy (EIS)

EIS is one of the most important electrochemical techniques where the impedance in a circuit is measured by ohms (as resistance unit). Over the other electrochemical technique, EIS offers several advantages reliant on the fact that it is a steady-state technique, that it utilizes small signal analysis, and that it is able to probe signal relaxations over a very wide range of applied frequency, from less than 1 mHz to greater than 1 MHz, using commercially available electrochemical working stations (potentiostat).

### C. Magnetic Purification

Magnetic separation is usually performed with magnetic carriers bearing an immobilized affinity or hydrophobic ligand or ion-exchange groups, or magnetic biopolymer particles having affinity to the isolated structure that are mixed with a sample containing target compound(s). Samples may be crude cell lysates, whole blood, plasma, ascites fluid, milk, whey, urine, cultivation media, wastes from food and fermentation industry and many others. Following an incubation period when the target compound(s) bind to the magnetic particles the whole magnetic complex is easily and rapidly removed from the sample using an appropriate magnetic separator. After washing out the contaminants, the isolated target compound(s) can be eluted and used for further work. Safarik et al., “Magnetic techniques for the isolation and purification of proteins and peptides” Biomagn Res Technol. 2004; 2:7.

Magnetic separation techniques have several advantages in comparison with standard separation procedures. This process is usually very simple, with only a few handling steps. All the steps of the purification procedure can take place in one single test tube or another vessel. There is no need for expensive liquid chromatography systems, centrifuges, filters, or other equipment. The separation process can be performed directly in crude samples containing suspended solid material. In some cases (e.g., isolation of intracellular proteins) it is even possible to integrate the disintegration and separation steps and thus shorten the total separation time. Due to the magnetic properties of magnetic adsorbents (and diamagnetic properties of majority of the contaminating molecules and particles present in the treated sample), they can be relatively easily and selectively removed from the sample. In fact, magnetic separation is the only feasible method for recovery of small magnetic particles (diameter ca 0.1-1 μm) in the presence of biological debris and other fouling material of similar size. Moreover, the power and efficiency of magnetic separation procedures is especially useful at large-scale operations. The magnetic separation techniques are also the basis of various automated procedures, especially magnetic particle-based immunoassay systems for the determination of a variety of analytes, among them proteins and peptides. Several automated systems for the separation of proteins or nucleic acids have become available recently.

Magnetic separation is usually very gentle to the target proteins or peptides. Even large protein complexes that tend to be broken up by traditional column chromatography techniques may remain intact when using the very gentle magnetic separation procedure. Both the reduced shearing forces and the higher protein concentration throughout the isolation process positively influence the separation process. Separation of target proteins using standard chromatography techniques often leads to the large volume of diluted protein solution. In this case appropriate magnetic particles can be used for their concentration instead of ultrafiltration, precipitation etc.

The basic equipment usually involves magnetic carriers with immobilized affinity or hydrophobic ligands, magnetic particles prepared from a biopolymer exhibiting affinity for the target compound(s) or magnetic ion-exchangers are usually used to perform the isolation procedure. Magnetic separators of different types can be used for magnetic separations, but many times cheap strong permanent magnets are equally efficient, especially in preliminary experiments.

Magnetic carriers and adsorbents can be either prepared in the laboratory, or commercially available ones can be used. Such carriers are usually available in the form of magnetic particles prepared from various synthetic polymers, biopolymers or porous glass, or magnetic particles based on the inorganic magnetic materials such as surface modified magnetite can be used. Many of the particles behave like superparamagnetic ones responding to an external magnetic field, but not interacting themselves in the absence of magnetic field. This is important due to the fact that magnetic particles can be easily resuspended and remain in suspension for a long time. In most cases, the diameter of the particles differs from approximately 50 nm to 10 μm. However, also larger magnetic affinity particles are also available, with the diameters up to millimeter range. Magnetic particles having the diameter larger than ca 1 μm can be easily separated using simple magnetic separators, while separation of smaller particles (magnetic colloids with the particle size ranging between tens and hundreds of nanometers) may require the usage of high gradient magnetic separators.

Commercially available magnetic particles can be obtained from a variety of companies. In most cases polystyrene is used as a polymer matrix, but carriers based on cellulose, agarose, silica, porous glass or silanized magnetic particles are also available.

### EXPERIMENTAL

### Example I

### Janus Particle Preparation

Janus particles were prepared by electron beam evaporation of chromium and gold unto a submonolayer of 5.1 μm red fluorescent carboxyl polystyrene (PS) particles (Magsphere®).

To prepare the submonolayer, two borosilicate glass slides were cleaned and dried with acetone (Millipore®; Sigma). One slide, the substrate slide, was mounted horizontally upon the immobile portion of a GenieTouch® syringe pump (Kent Scientific), while the other slide, the deposition slide, was mounted upon the moving portion of the pump connected to the driveshaft. The deposition slide was brought into contact with the stationary slide at a roughly 45° angle. The particles were washed 4 times with milli-Q water (MQW) to remove surfactants and concentrate the solution to 10 wt. %. After briefly sonicating and vortexing the particle solution, 10 μL of the prepared particle solution was pipetted onto the substrate slide at the point of contact with the deposition slide, forming a thin, uniform line across the contact line between the slides.

The syringe pump was set to the full position for a 30 mL Becton Dickinson syringe, and the deposition slide was pushed across the substrate slide at a set “speed” of 2.5 mL min-1 until the deposited particle solution was exhausted approximately half-way across the deposition slide. This corresponded to an “ejected volume” of 13 mL as indicated by the syringe pump. The deposition slide was then cleaned to prevent particle buildup after which the slides were once again brought into contact slightly past where the previous deposition ended. This procedure was repeated to fill the slide.

Slides with submonolayers were then coated with a 10 nm layer of chromium, for increased metal patch adhesion, and a 30 nm layer of gold using an electron-beam evaporator (Edwards Auto 306 Cryo®). The coated particles were then removed from their slides by gently scraping them from the slide with a metal spatula, resuspending in MWQ, and transferring into a microcentrifuge tube. Finally, the particles were sonicated for 30-60 seconds to break up particle aggregates.

### Example II

### Janus Particle Or ICEM Washing

To wash Janus particles or ICEMs, they were first resuspended in MQW or PBS with 0.02 vol. % polysorbate 20 (Tween 20, Sigma Aldrich) to aid in pelleting and subsequently centrifuged at 10,000×G for 6 minutes. The 0.02 vol. % Tween 20 was excluded only when washing particles into pure MQW immediately before i) submonolayer deposition or ii) propulsion experiments. Particles were typically washed 3-4 times between experimental steps

### Example III

### Conjugation of mPEG-Amine to Janus Particles

Janus particles were functionalized with PEG on their PS hemisphere using simple carbodiimide chemistry.

Typically, 5×10⁶ Janus particles were resuspended in 110 μL of a solution of 10 mg mL-1 1 kDa methoxypolyethylene glycol amine (mPEG-amine, Alfa Aesar) in a 0.05 M 2-(N-morpholino) ethanesulfonic acid monohydrate (MES, MP Biomedicals) buffer containing 100 mg mL-1 1-ethyl-3-(3-dimethylaminopropyl) carbodiimide hydrochloride (EDC, Chem Impex) at pH=5.0. This solution was then incubated for 2 hours at ambient temperature in a VorTemp 56® shaking incubator (Labnet) at 900-1100 rpm. The particles were occasionally redispersed if settling was noted. At the end of the incubation time, the solution was washed 3-4 times with 1×PBS ((VWR Life Science) with 0.02% Tween 20.

### Example IV

### Conjugation of Biotin-PEG-Thiol to Janus Particles to Prepare ICEMs

To conjugate biotin-PEG-thiol (BPT, Biopharma PEG) to the gold hemisphere of the Janus particles to prepare ICEMs, 5×10⁶ Janus particles with PS-conjugated PEG were resuspended in 110 μL of a solution of 10 mg mL⁻¹ 1 kDa BPT in PBS with 3 mM ethylenediaminetetraacetic acid (EDTA, Sigma) at pH=8. This solution was incubated overnight at ambient temperature in a shaking incubator at 900-1100 rpm. At the end of the incubation time, the solution was typically washed three times in MWQ with 0.02% Tween20. With this step, the ICEMs were fully functionalized.

### Example V

### Biomolecule Capture by ICEMs

Streptavidin, Alexa Fluor 594-conjugated streptavidin (Invitrogen Thermo Scientific), or free biotin (D-(+)-biotin (Research Products International Corporation) was diluted with PBS to 100 μL at the desired concentration, depending on experimental requirements. Then, 5×10⁵ ICEMs were resuspended in the SA solution and allowed to incubate at ambient temperature for 30 minutes in a shaking incubator at 900-1100 rpm. At the end of the incubation time, each condition was washed 3-4 times with MQW, after which each ICEM condition was resuspended in 200-500 μL MQW.

### Example VI

### Streptavidin (SA) Blocking

To create a blocked streptavidin (SA) control, free biotin was dissolved in deionized water (DIW) and added to a solution of SA in a roughly 500-fold molar excess. The solution was then allowed to incubate for 75 minutes.

### Example VII

### Coplanar Propulsion Chamber Fabrication

To fabricate coplanar propulsion chambers, borosilicate glass slides were masked with a roughly 2 mm paper or adhesive tape that covered the length of the slides. Then, electron-beam evaporation was used to deposit 10-30 nm of chromium and 100 nm of gold on the masked slides. After removal of the mask, a roughly 2 mm uncoated gap in the center of the slide along its length remained, with two electrodes on either side. Two strips of copper tape were applied on top of the gold electrodes to form connection points for electrical leads.

### Example VIII

### ICEM Propulsion Experiments

Coplanar propulsion chambers were washed thoroughly with acetone before each experiment. A hydrophobic barrier pen (IHC World) was used to draw a roughly 1.25 cm×1.25 cm square outline overlapping the two electrodes. A mask defined with two pieces of polytetrafluorethylene (PTFE) tape (RS Crum & Company) was placed perpendicular to the electrodes at the boarder of the hydrophobic boundary, leaving a small rectangular space between the electrodes. A 10-20 μL aliquot of Janus particles or ICEMs were pipetted onto the small space left between the PTFE tape spacers and gold electrodes, and a coverslip was placed over the sample and spacers.

The loaded propulsion chamber was placed onto a Zeiss Axio Vert® A1 TL/RL inverted fluorescence microscope equipped with a Axiocam® 305 mono camera (Zeiss, Germany). An Agilent 33210A waveform generator (Agilent) was attached to a Tegam 2340 high voltage amplifier (Tegam), from which electrical leads were attached to the copper tape affixed to the propulsion chamber electrodes. A 4 kHz square wave AC signal was generated, amplified, and applied to the propulsion chamber, forming an electric field with magnitudes that ranged from 500-1000 V cm⁻¹. Resultant particle motion was observed and recorded using the microscope camera, typically in fluorescence mode.

### Example IX

### Particle Speed Analysis

Video recordings of ICEMs undergoing ICEP captured during experiments were analyzed using ImageJ® software. Videos were exported as AVI files while maintaining the native micron/pixel ratio and relative time information. Using the ImageJ® software, each frame was converted to a binary image in a stack, and individual particle positions in each frame were extracted by use of either the “analyze particles” or “Trackmate” options in the software. The linear speed of each particle was then calculated using this extracted data. Only speeds of ICEMs that traveled linearly within 15° of normal to the gold electrodes were recorded and analyzed.

### Example X

### Four Parameter Logistic Fitting

ICEM speed data was fit with a four-parameter logistic (4PL) curve to visualize ICEM response and determine maximum and minimum response values. To do so, experimental data (e.g., from 0-2×10⁴ nM SA, See, FIG. 5B) was fit to the standard 4PL equation, given by:

$$y = b + \frac{a - b}{1 + {( \frac{x}{c} )}^{d}}$$

where y is ICEM speed, x is SA concentration, c is the mid-range concentration, d is the slope factor, and a and b are ICEM speed or response at the minimum and maximum SA concentrations, respectively. MATLAB was used for all fitting. Extracted parameter values were a=11.51 μm sec⁻¹, b=6.20 μm sec⁻¹, c=2.70, and b=2.18. Using these values, a roughly 46% decrease in ICEM speed at the maximum response was determined.

### Example XI

### EAP Biosensor Sensitivity

Gold patches on the particles are modified with antibodies to capture an OPG biomarker, which is an abundant plasma protein in the serum of some individuals with breast cancer (40-160 ng/ml). Odén et al., “Plasma osteoprotegerin and breast cancer risk in BRCA1 and BRCA2 mutation carriers” Oncotarget 2016, 7(52), 86687-86694. Known levels of OPG spiked into blood enable calibrates particle velocity for different antibody concentrations at fixed electric field strengths and frequencies. The LOD and dynamic range of the biosensors are determined in whole blood at each coating density. A second set of particles are modified to detect HER2, a less abundant protein (12-20 ng/ml). Sauter, E. R., “Reliable Biomarkers to Identify New and Recurrent Cancer” Eur J Breast Health 2017, 13(4), 162-167. The data is validated against a conventional ELISA.

### Example XII

### Efficacy of EAP Biosensors to Detect A Multi-Biomarker Panel

EAPs of various shapes encode for (i.e., provides an identification) a unique biomarker. Each particle design is individually calibrated to detect biomarkers of various morphology (e.g., by variations in angular structure): for example, OPG, HER2, cell-free DNA (cfDNA), interleukin-6 (IL-6), CD44⁺ exosomes, and circulating tumor cells (CTCs) are all attached to EAPs having differences in angular structure. See, FIG. 13.

After calibration, all six biomarkers are detected in a single assay at their physiological concentrations in blood samples from a late-stage breast cancer patient. The shape-encoding comparison also assesses the feasibility of expanding the dynamic range of the biosensors.

The EAP biosensors are validated by detecting CD44+ exosomes, broad-panel cfDNA, IL-6, CTCs, HER2 and OPG at physiological levels from blood in a single assay. All six biomarkers will be doped into blood at levels corresponding to patients with advanced breast cancer: i) CD44+ exosomes—1×10⁶ in 1 mL; ii) cfDNA—10 ng/ml; iii) IL-6—25 pg/mL; iv) CTCs—1 cell in 1 mL of blood; v) HER2—20 ng/ml; and vi) OPG—40 ng/mL.

### Example XIII

### Biomarker Binding Kinetics

The example measures the relationship between incubation time and assay sensitivity with the following method:
v) Add EAPs to a biomarker (e.g., OPG) solution (buffer or blood), incubate, mix via rotation for 20-120 min.
ii) Dilute with red blood cell (RBC) lysis buffer, separate via magnetic column for 3 min.
iii) Dilute EAPs, separate via magnetic column again to purify for 3 min.
iv) Add EAPs to electrokinetic chamber, apply an AC field for 2 min.
v) Video-record EAP propulsion via basic microscopy for 2 min.

The sensitivity of the EAPs to OPG are measured as a function of incubation time (20-120 min). Capture performances are compared head-to-head against ELISA. This workflow, up to 10-fold faster than ELISA (˜0.5 vs. 5 hrs), is made possible by the use of magnetic separation, which obviates the need for centrifugation. Suspended particles also accelerate the workflow by reducing the length scales required for binding. Lee et al., “Accelerated immunoassays based on magnetic particle dynamics in a rotating capillary tube with stationary magnetic field” Anal Chem 2012, 84 (19), 8317-22; and Rettcher et al., “Simple and portable magnetic immunoassay for rapid detection and sensitive quantification of plant viruses” Appl Environ Microbiol 2015, 81(9), 3039-48. As soon as EAPs are washed, AC current stimulation provides quantitative information through particle tracking.

### Example IVX

### EAP Detection of Osteoprotegerin

Osteoprotegerin (OPG) will be spiked into purified buffer to calibrate EAP propulsion with OPG concentration. For each condition, the number of analyzed EAP trajectories achieves a statistical significance of at least 3 standard deviations above the mean. Lozano et al., “Difference Between Analytical Sensitivity and Detection Limit. American Journal of Clinical Pathology 1997, 107(5), 619.

Briefly, human OPG (TNFRSF11B) is spiked into porcine blood. The coating density of anti-OPG antibodies on the EAPs is varied using conventionally described methods described earlier. Oliveira et al., “Impact of conjugation strategies for targeting of antibodies in gold nanoparticles for ultrasensitive detection of 17beta-estradiol” Sci Rep 2019, 9 (1), 13859. Coating densities are verified by the use of fluorescent antibodies, where the number of antibodies per particle will be measured by fluorescence inspection (plate reader or flow cytometer) to enumerate molecules equivalent of antibody. Shields et al., “Nucleation and growth synthesis of siloxane gels to form functional, monodisperse, and acoustically programmable particles” Angew Chem Int Ed Engl 2014, 53(31), 8070-3.

The LOD and dynamic range will be tested for each coating density in blood using the aforementioned calibration studies and validated by comparison to a commercial ELISA (e.g., Thermo Fisher; LOD of 1 μg/mL and dynamic range up to 0.9 ng/ml).

### Example XV

### EAP Detection of Human Epidermal Growth Factor Receptor 2

HER2 concentrations in pre-test blood samples is determined by ELISA because healthy blood contains ˜12 ng/mL HER2. This permits an appropriate amount of HER2 to be spiked into each blood sample so that the final amount of HER2 is consistent across all groups.

Calibration studies will be performed with known amounts of HER2 in each blood sample and measuring resultant EAP velocities. The coating density of anti-HER2 antibodies will be varied using methods described above. The LOD and dynamic range for each coating density will be determined and validated against a commercial ELISA (e.g., ThermoFisher; LOD of 8 μg/mL and dynamic range up to 2 ng/ml).

### Example XVI

### EAP Biosensor Dynamic Range Determination

EAPs with different shapes will be prepared where each differently shaped EAP is coated with a different antibody coating density. For example, the EAPs are coated with OPG concentrations ranging between 1.0-10⁶ pg/mL. The dynamic ranges of each EAP/OPG coating density will be tested and compared to single particle controls and validated by ELISA.

### REFERENCES

(1) Ranjan, P.; Parihar, A.; Jain, S.; Kumar, N.; Dhand, C.; Murali, S.; Mishra, D.; Sanghi, S. K.; Chaurasia, J. P.; Srivastava, A. K.; Khan, R. Biosensor-Based Diagnostic Approaches for Various Cellular Biomarkers of Breast Cancer: A Comprehensive Review. Anal. Biochem. 2020, 610, 113996. https://doi.org/10.1016/j.ab.2020.113996.
(2) Wolkowicz, K. L.; Aiello, E. M.; Vargas, E.; Teymourian, H.; Tehrani, F.; Wang, J.; Pinsker, J. E.; Doyle, F. J.; Patti, M.; Laffel, L. M.; Dassau, E. A Review of Biomarkers in the Context of Type 1 Diabetes: Biological Sensing for Enhanced Glucose Control. Bioeng. Transl. Med. 2020, 6 (2), e10201. https://doi.org/10.1002/btm2.10201.
(3) Justino, C. I. L.; Duarte, A. C.; Rocha-Santos, T. A. P. Recent Progress in Biosensors for Environmental Monitoring: A Review. Sensors 2017, 17 (12), 2918. https://doi.org/10.3390/s17122918.
(4) Naresh, V.; Lee, N. A Review on Biosensors and Recent Development of Nanostructured Materials-Enabled Biosensors. Sensors 2021, 21 (4), 1109. https://doi.org/10.3390/s21041109.
(5) Saylan, Y.; Erdem, Ö.; Ünal, S.; Denizli, A. An Alternative Medical Diagnosis Method: Biosensors for Virus Detection. Biosensors 2019, 9 (2), 65. doi.org/10.3390/bios9020065. 20
(6) Polat, E. O.; Cetin, M. M.; Tabak, A. F.; Bilget Güven, E.; Uysal, B. Ö.; Arsan, T.; Kabbani, A.; Hamed, H.; Gül, S. B. Transducer Technologies for Biosensors and Their Wearable Applications. Biosensors 2022, 12 (6), 385. doi.org/10.3390/bios12060385.
(7) Pacheco, M.; López, M. Á.; Jurado-Sánchez, B.; Escarpa, A. Self-Propelled Micromachines for Analytical Sensing: A Critical Review. Anal. Bioanal. Chem. 2019, 411 (25), 6561-6573. doi.org/10.1007/s00216-019-02070-z.
(8) Maria-Hormigos, R.; Jurado-Sánchez, B.; Escarpa, A. Biocompatible Micromotors for Biosensing. Anal. Bioanal. Chem. 2022, 414 (24), 7035-7049. doi.org/10.1007/s00216-022-04287-x.
(9) Jurado-Sánchez, B.; Campuzano, S.; Pingarrón, J. M.; Escarpa, A. Janus Particles and Motors: Unrivaled Devices for Mastering (Bio) Sensing. Microchim. Acta 2021, 188 (12), 416. doi.org/10.1007/s00604-021-05053-z.
(10) Yánez-Sedeño, P.; Campuzano, S.; Pingarrón, J. M. Janus Particles for (Bio) Sensing. Appl. Mater. Today 2017, 9, 276-288. doi.org/10.1016/j.apmt.2017.08.004.
(11) Molinero-Fernández, Á.; Arruza, L.; López, M. Á.; Escarpa, A. On-the-Fly Rapid Immunoassay for Neonatal Sepsis Diagnosis: C-Reactive Protein Accurate Determination Using Magnetic Graphene-Based Micromotors. Biosens. Bioelectron. 2020, 158, 112156. doi.org/10.1016/j.bios.2020.112156.
(12) Lu, C.; Liu, X.; Li, Y.; Yu, F.; Tang, L.; Hu, Y.; Ying, Y. Multifunctional Janus Hematite-Silica Nanoparticles: Mimicking Peroxidase-Like Activity and Sensitive Colorimetric Detection of Glucose. ACS Appl. Mater. Interfaces 2015, 7 (28), 15395-15402. doi.org/10.1021/acsami.5b03423.
(13) Hosseini, S.; Vázquez-Villegas, P.; Rito-Palomares, M.; Martinez-Chapa, S. O. Advantages,

Disadvantages and Modifications of Conventional ELISA. In Enzyme-linked Immunosorbent Assay (ELISA): From A to Z; Hosseini, S., Vázquez-Villegas, P., Rito-Palomares, M., Martinez-Chapa, S. O., Eds.; SpringerBriefs in Applied Sciences and Technology; Springer: Singapore, 2018; pp 67-115. doi.org/10.1007/978-981-10-6766-2_5.
(14) Paniagua, G.; Villalonga, A.; Eguílaz, M.; Vegas, B.; Parrado, C.; Rivas, G.; Díez, P.; Villalonga, R. Amperometric Aptasensor for Carcinoembryonic Antigen Based on the Use of Bifunctionalized Janus Nanoparticles as Biorecognition-Signaling Element. Anal. Chim. Acta 2019, 1061, 84-91. doi.org/10.1016/j.aca.2019.02.015.
(15) Yang, Y.-J.; Zhou, Y.; Xing, Y.; Zhang, G.-M.; Zhang, Y.; Zhang, C.-H.; Lei, P.; Dong, C.; Deng, X.; He, Y.; Shuang, S. A Label-Free Aptasensor Based on Aptamer/NH2 Janus Particles for Ultrasensitive Electrochemical Detection of Ochratoxin A. Talanta 2019, 199, 310-316. doi.org/10.1016/j.talanta.2019.02.015.
(16) María-Hormigos, R.; Molinero-Fernández, Á.; López, M. Á.; Jurado-Sánchez, B.; Escarpa, A. Prussian Blue/Chitosan Micromotors with Intrinsic Enzyme-like Activity for (Bio)-Sensing Assays. Anal. Chem. 2022, 94 (14), 5575-5582. doi.org/10.1021/acs.analchem.1c05173.
(17) Jurado-Sánchez, B.; Pacheco, M.; Rojo, J.; Escarpa, A. Magnetocatalytic Graphene Quantum Dots Janus Micromotors for Bacterial Endotoxin Detection. Angew. Chem. Int. Ed. 2017, 56 (24), 6957-6961. doi.org/10.1002/anie.201701396.
(18) Li, J.; Concellon, A.; Yoshinaga, K.; Nelson, Z.; He, Q.; Swager, T. M. Janus Emulsion Biosensors for Anti-SARS-COV-2 Spike Antibody. ACS Cent. Sci. 2021, 7 (7), 1166-1175. doi.org/10.1021/acscentsci.1c00173. 21
(19) Wu, J.; Balasubramanian, S.; Kagan, D.; Manesh, K. M.; Campuzano, S.; Wang, J. Motion-Based DNA Detection Using Catalytic Nanomotors. Nat. Commun. 2010, 1, 36. doi.org/10.1038/ncomms1035.
(20) Nguyen, K. V.; Minteer, S. D. DNA-Functionalized Pt Nanoparticles as Catalysts for Chemically Powered Micromotors: Toward Signal-on Motion-Based DNA Biosensor. Chem. Commun. 2015, 51 (23), 4782-4784. doi.org/10.1039/C4CC10250A.
(21) Yu, X.; Li, Y.; Wu, J.; Ju, H. Motor-Based Autonomous Microsensor for Motion and Counting Immunoassay of Cancer Biomarker. Anal. Chem. 2014, 86 (9), 4501-4507. doi.org/10.1021/ac500912c.
(22) Draz, M. S.; Lakshminaraasimulu, N. K.; Krishnakumar, S.; Battalapalli, D.; Vasan, A.; Kanakasabapathy, M. K.; Sreeram, A.; Kallakuri, S.; Thirumalaraju, P.; Li, Y.; Hua, S.; Yu, X. G.; Kuritzkes, D. R.; Shafiee, H. Motion-Based Immunological Detection of Zika Virus Using Pt-Nanomotors and a Cellphone. ACS Nano 2018, 12 (6), 5709-5718. doi.org/10.1021/acsnano.8b01515.
(23) Yuan, K.; Cuntín-Abal, C.; Jurado-Sánchez, B.; Escarpa, A. Smartphone-Based Janus Micromotors Strategy for Motion-Based Detection of Glutathione. Anal. Chem. 2021, 93 (49), 16385-16392. doi.org/10.1021/acs.analchem.1c02947.
(24) Russell, S. M.; Alba-Patiño, A.; Borges, M.; de la Rica, R. Multifunctional Motion-to-Color Janus Transducers for the Rapid Detection of Sepsis Biomarkers in Whole Blood. Biosens. Bioelectron. 2019, 140, 111346. doi.org/10.1016/j.bios.2019.111346.
(25) Zhang, X.; Chen, C.; Wu, J.; Ju, H. Bubble-Propelled Jellyfish-like Micromotors for DNA Sensing. ACS Appl. Mater. Interfaces 2019, 11 (14), 13581-13588. doi.org/10.1021/acsami.9b00605.
(26) Boymelgreen, A.; Yossifon, G. Observing Electrokinetic Janus Particle-Channel Wall Interaction Using Microparticle Image Velocimetry. Langmuir 2015, 31 (30), 8243-8250. doi.org/10.1021/acs.langmuir.5b01199.
(27) Harraq, A. A.; Choudhury, B. D.; Bharti, B. Field-Induced Assembly and Propulsion of Colloids. Langmuir 2022, 38 (10), 3001-3016. doi.org/10.1021/acs.langmuir.1c02581.
(28) Peng, C.; Lazo, I.; Shiyanovskii, S. V.; Lavrentovich, O. D. Induced-Charge Electro-Osmosis around Metal and Janus Spheres in Water: Patterns of Flow and Breaking Symmetries. Phys. Rev. E 2014, 90 (5), 051002. doi.org/10.1103/PhysRevE.90.051002.
(29) Gangwal, S.; Cayre, O. J.; Bazant, M. Z.; Velev, O. D. Induced-Charge Electrophoresis of Metallodielectric Particles. Phys. Rev. Lett. 2008, 100 (5), 058302. doi.org/10.1103/PhysRevLett.100.058302.
(30) Bazant, M. Z.; Squires, T. M. Induced-Charge Electrokinetic Phenomena: Theory and Microfluidic Applications. Phys. Rev. Lett. 2004, 92 (6), 066101. doi.org/10.1103/PhysRevLett.92.066101.
(31) Brooks, A. M.; Sabrina, S.; Bishop, K. J. M. Shape-Directed Dynamics of Active Colloids Powered by Induced-Charge Electrophoresis. Proc. Natl. Acad. Sci. 2018, 115 (6), E1090-E1099. doi.org/10.1073/pnas.1711610115.
(32) Katuri, J.; Poehnl, R.; Sokolov, A.; Uspal, W.; Snezhko, A. Arrested-Motility States in Populations of Shape-Anisotropic Active Janus Particles. Sci. Adv. 2022, 8 (26), eabo3604. doi.org/10.1126/sciadv.abo3604. 22
(33) Wang, Z.; Wang, Z.; Li, J.; Cheung, S. T. H.; Tian, C.; Kim, S.-H.; Yi, G.-R.; Ducrot, E.; Wang, Y. Active Patchy Colloids with Shape-Tunable Dynamics. J. Am. Chem. Soc. 2019, 141 (37), 14853-14863. doi.org/10.1021/jacs.9b07785.
(34) Lee, J. G.; Brooks, A. M.; Shelton, W. A.; Bishop, K. J. M.; Bharti, B. Directed Propulsion of Spherical Particles along Three Dimensional Helical Trajectories. Nat. Commun. 2019, 10 (1), 2575. doi.org/10.1038/s41467-019-10579-1.
(35) Lee, J. G.; Al Harraq, A.; Bishop, K. J. M.; Bharti, B. Fabrication and Electric Field-Driven Active Propulsion of Patchy Microellipsoids. J. Phys. Chem. B 2021, 125 (16), 4232-4240. doi.org/10.1021/acs.jpcb.1c01644.
(36) Wang, Z.; Mu, Y.; Lyu, D.; Wu, M.; Li, J.; Wang, Z.; Wang, Y. Engineering Shapes of Active Colloids for Tunable Dynamics. Curr. Opin. Colloid Interface Sci. 2022, 61, 101608. doi.org/10.1016/j.cocis.2022.101608.
(37) Park, S.; Yossifon, G. Micromotor-Based Biosensing Using Directed Transport of Functionalized Beads. ACS Sens. 2020, 5 (4), 936-942. doi.org/10.1021/acssensors.9b02041.
(38) Pascall, A. J.; Squires, T. M. Induced Charge Electro-Osmosis over Controllably Contaminated Electrodes. Phys. Rev. Lett. 2010, 104 (8), 088301. doi.org/10.1103/PhysRevLett.104.088301.
(39) M.Boymelgreen, A.; Kunti, G.; Garcia-Sanchez, P.; Ramos, A.; Yossifon, G.; Miloh, T. The Role of Particle-Electrode Wall Interactions in Mobility of Active Janus Particles Driven by Electric Fields. J. Colloid Interface Sci. 2022, 616, 465-475. doi.org/10.1016/j.jcis.2022.02.017.
(40) Viefhues, M.; Manchanda, S.; Chao, T.-C.; Anselmetti, D.; Regtmeier, J.; Ros, A. Physisorbed Surface Coatings for Poly(Dimethylsiloxane) and Quartz Microfluidic Devices. Anal. Bioanal. Chem. 2011, 401 (7), 2113. doi.org/10.1007/s00216-011-5301-z.
(41) Paratore, F.; Boyko, E.; Kaigala, G. V.; Bercovici, M. Electroosmotic Flow Dipole: Experimental Observation and Flow Field Patterning. Phys. Rev. Lett. 2019, 122 (22), 224502. doi.org/10.1103/PhysRevLett.122.224502.
(42) Honegger, T.; Peyrade, D. Dielectrophoretic Properties of Engineered Protein Patterned Colloidal Particles. Biomicrofluidics 2012, 6 (4), 044115. doi.org/10.1063/1.4771544.
(43) Pawar, A. B.; Kretzschmar, I. Fabrication, Assembly, and Application of Patchy Particles. Macromol. Rapid Commun. 2010, 31 (2), 150-168. doi.org/10.1002/marc.200900614.
(44) Prevo, B. G.; Kuncicky, D. M.; Velev, O. D. Engineered Deposition of Coatings from Nanoand Micro-Particles: A Brief Review of Convective Assembly at High Volume Fraction. Colloids Surf. Physicochem. Eng. Asp. 2007, 311 (1), 2-10. doi.org/10.1016/j.colsurfa.2007.08.030.
(45) Shields IV, C. W.; Han, K.; Ma, F.; Miloh, T.; Yossifon, G.; Velev, O. D. Supercolloidal Spinners: Complex Active Particles for Electrically Powered and Switchable Rotation. Adv. Funct. Mater. 2018, 28 (35), 1803465. doi.org/10.1002/adfm.201803465.
(46) Al Harraq, A.; Bello, M.; Bharti, B. A Guide to Design the Trajectory of Active Particles: From Fundamentals to Applications. Curr. Opin. Colloid Interface Sci. 2022, 61, 101612. doi.org/10.1016/j.cocis.2022.101612.
(47) Dai, Y.; Liu, C. C. Recent Advances on Electrochemical Biosensing Strategies toward Universal Point-of-Care Systems. Angew. Chem. Int. Ed. 2019, 58 (36), 12355-12368. doi.org/10.1002/anie.201901879. 23
(48) Zhuang, Y.; Zhu, Q.; Tu, C.; Wang, D.; Wu, J.; Xia, Y.; Tong, G.; He, L.; Zhu, B.; Yan, D.; Zhu, X. Protein Resistant Properties of Polymers with Different Branched Architecture on a Gold Surface. J. Mater. Chem. 2012, 22 (45), 23852-23860. doi.org/10.1039/C2JM34306A.
(49) Thorek, D. L. J.; Elias, D. R.; Tsourkas, A. Comparative Analysis of Nanoparticle-Antibody Conjugations: Carbodiimide versus Click Chemistry. Mol. Imaging 2009, 8 (4), 221-229.
(50) Upadhyayula, S.; Quinata, T.; Bishop, S.; Gupta, S.; Johnson, N. R.; Bahmani, B.; Bozhilov, K.; Stubbs, J.; Jreij, P.; Nallagatla, P.; Vullev, V. I. Coatings of Polyethylene Glycol for Suppressing Adhesion between Solid Microspheres and Flat Surfaces. Langmuir 2012, 28 (11), 5059-5069. doi.org/10.1021/la300545v.
(51) Haugland, R. P.; You, W. W. Coupling of Antibodies With Biotin. In Avidin-Biotin Interactions: Methods and Applications; McMahon, R. J., Ed.; Methods In Molecular Biology™; Humana Press: Totowa, NJ, 2008; pp 13-23. doi.org/10.1007/978-1-59745-579-4_2.
(52) Li, L.; Shields, C. W.; Huang, J.; Zhang, Y.; Ohiri, K. A.; Yellen, B. B.; Chilkoti, A.; López, G. P. Rapid Capture of Biomolecules from Blood via Stimuli-Responsive Elastomeric Particles for Acoustofluidic Separation. Analyst 2021, 145 (24), 8087-8096. doi.org/10.1039/DOAN01164A.
(53) Weimer, B. C.; Walsh, M. K.; Wang, X. Influence of a Poly-Ethylene Glycol Spacer on Antigen Capture by Immobilized Antibodies. J. Biochem. Biophys. Methods 2000, 45 (2), 211-219. doi.org/10.1016/S0165-022X(00)00114-7.
(54) O'Connell, M. A.; Belanger, B. A.; Haaland, P. D. Calibration and Assay Development Using the Four-Parameter Logistic Model. Chemom. Intell. Lab. Syst. 1993, 20 (2), 97-114. doi.org/10.1016/0169-7439(93)80008-6.

1. A composition comprising:
i) a plurality of first electrokinetic active particles (EAPs) having a first shape with a bound first biomolecule recognition element; and
ii) a plurality of second EAPs having a second shape with a bound second biomolecule recognition element.

2. (canceled)

3. The composition of claim 1, wherein said first and second biomolecule recognition element has a binding affinity to a biomarker.

4. (canceled)

5. The composition of claim 1, wherein said first shape has an angular structure that is different than said second shape.

6. The composition of claim 5, wherein said angular structure of the first shape comprises an angle that is less than that of the second shape.

7. (canceled)

8. The composition of claim 1, wherein said first biomolecule recognition element has a different coating density than said second biomolecule recognition element.

9. The composition of claim 1, wherein each of said plurality of first and second EAPs further comprise a dielectric metallic surface region.

10. (canceled)

11. The composition of claim 9, wherein said metallic surface region is selected from the group consisting of magnetic or non-magnetic metals or conductive polymers.

12-13. (canceled)

14. The composition of claim 1, wherein said first and second EAPs are selected from the group consisting of microparticles, nanoparticles and Janus particles.

15-18. (canceled)

19. The composition of claim 9, wherein said metallic surface further comprises a layer selected from the group consisting of a chromium layer, an indium tin oxide electrically conducting layer and a chromium and gold layer.

20-25. (canceled)

26. A method, comprising:
a) providing a solution comprising;
i) a plurality of first electrokinetic active particles (EAPs) having a first shape and attached to a first biomolecule recognition element;
ii) a plurality of second EAPs having a second shape and attached to a second biomolecule recognition element;
iii) an electrokinetic propulsion chamber comprising a coplanar or non-coplanar electrode pair separated by an electrically insulated region; and
iv) a camera mounted to a magnifying lens;
b) placing an aliquot of said solution on said electrokinetic propulsion chamber;
c) applying an alternating current electric field to said electrokinetic propulsion chamber;
d) recording a series of images or video of said solution with said camera; and
e) determining an electrokinetic motion of said first and second EAPs from said image

27. The method of claim 26, wherein said first biomolecule recognition element is specifically bound to a first biomarker and said second biomolecule recognition element is specifically bound to a second biomarker, wherein said first biomarker is different from said second biomarker.

28-29. (canceled)

30. The method of claim 26, wherein said motion is an electrokinetic speed or acceleration of said first EAP being slower than said second EAP.

31. The method of claim 26, wherein said motion is an electrokinetic speed or acceleration of said first EAP being faster than said second EAP.

32-33. (canceled)

34. The method of claim 26, wherein said method further comprises purifying said first EAP from said second EAP.

35. (canceled)

36. The method of claim 26, wherein said method further comprises identifying and quantifying said first and second biomarkers.

37-41. (canceled)

42. The method of claim 26, wherein said first shape has an angular structure that is different than said second shape.

43. The method of claim 26, wherein said angular structure of said first shape comprises an angle that is less than that of the second shape.

44-45. (canceled)

46. The method of claim 26, wherein each of said plurality of first and second EAPs further comprise a dielectric metallic surface region.

47. (canceled)

48. The method of claim 46, wherein said metallic surface region is selected from the group consisting of magnetic metals, non-magnetic metals and conductive polymers.

49-52. (canceled)

53. The method of claim 46, wherein said metallic surface region comprises a layer selected from the group consisting of a chromium layer, an indium tin oxide layer and a chromium and gold layer.

54-109. (canceled)
